(19) United States
(12) Patent Application Publication
(10) Pub. No.: US 2004/0180370 A1
(43) Pub. Date: Sep. 16, 2004

(54) Genetic diagnosis of alcoholism subtypes

(75) Inventors: Boris Tabakoff, Denver, CO (US); Larry Martinez, Colorado Springs, CO (US); Paula Hoffman, Denver, CO (US)

Correspondence Address: Christine A. Lekutis, MEDLEN & CARROLL, LLP, 101 Howard Street, Suite 350, San Francisco, CA, 94105, US

(73) Assignee: THE REGENTS OF THE UNIVERSITY OF COLORADO, a body corporate, Boulder, CA

(21) Appl. No.: 10/766,590

(22) Filed: Jan. 27, 2004

Related U.S. Application Data
(60) Provisional application No. 60/443,072, filed on Jan. 27, 2003.

(51) Int. Cl. C12Q 1/68; C12P 19/34

(52) U.S. Cl. 435/6; 435/91.2

(57) ABSTRACT

The present invention relates to compositions and methods for determining whether an individual is predisposed to or protected from alcoholism. In particular, the present invention provides genetic markers useful for the diagnosis, characterization, and treatment or prevention of alcohol tolerance, abuse and/or dependence.

This application claims the benefit of U.S. Provisional Application No. 60/443,072, filed on Jan. 27, 2003.

The invention was made in part with Government support, by the National Institutes of Health, National Institute on Alcohol Abuse and Alcoholism Grant RO1AA13162. As such, the Government has certain rights in the invention.

### FIELD OF THE INVENTION

The present invention relates to compositions and methods for determining whether an individual is predisposed to or protected from alcoholism. In particular, the present invention provides genetic markers useful for the diagnosis, characterization, and treatment or prevention of alcohol tolerance, abuse and/or dependence. In addition, the present invention provides targets for drug development for the treatment of alcoholism.

### BACKGROUND OF THE INVENTION

Alcoholism is a term used in lay language to refer to both alcohol abuse and alcohol dependence. Alcohol abuse is a disorder characterized by a pathological pattern of alcohol use leading to significant social, occupational, and/or medical impairments. Alcohol dependence is defined as a maladaptive pattern of alcohol use that includes three or more of the following problems: 1) physiologic tolerance; 2) withdrawal symptoms; 3) use in excess of intentions; 4) desire or unsuccessful efforts to limit use; 5) much time spent in acquiring, using alcohol, or recovering from use; 6) reduction in important social, occupational, or recreational activities; and 7) continued use despite knowledge of having a persistent or recurrent physical or psychological problem that is exacerbated by alcohol use (See, e.g., O'Connor, “Alcohol Abuse and Dependency,” in Scientific American Medicine, vol. 3, chapter 13, pp. 1-9, 2000; and Diagnostic and Statistical Manual of Mental Disorders, 4th edition, American Psychiatric Association, Washington D.C., 1994).

In the United States, the prevalence of alcohol dependence is around 6% for men and 2% for women (Caetano and Cunradi, Addiction, 97:633-645, 2002). Nearly 100,000 Americans die each year as a result of alcohol abuse, and alcohol is a factor in more than half of the country's homicides, suicides, and traffic accidents. Thus, alcohol abuse and dependence are costly to society.

Treatment of alcohol dependence is directed to helping the patient to permanently stop or diminish drinking alcohol. While self-help groups such as Alcoholics Anonymous are beneficial to some patients, others may require medication and/or clinical care. In any event, the first step in this process is detoxification. Patients undergoing alcohol withdrawal may experience symptoms such as anxiety attacks, shakes, chills or sweats, chest pain, headache, nausea or vomiting, abdominal pain, delusions, and hallucinations, within one to three days of abstinence. Patients presenting with symptoms of severe withdrawal (e.g., delerium tremens) are typically given tranquilizers (e.g., diazepam, lorezepam, or librium) and placed under observation in a hospital setting. The second step in this process is the maintenance of abstinence. In some instances, medications such as naltrexone or antabuse may be prescribed to lessen a patient's craving for alcohol. Unfortunately, the medications currently available for this purpose are not highly efficacious.

Family, twin and adoption studies provide convincing evidence of the existence of a strong genetic component to the risk of alcohol abuse and dependence (Schuckit, Am J Addict, 9:103, 2000). In fact, multiple putative alcoholism vulnerability genes have been identified including: D2 dopamine-receptor gene (DRD2), alcohol dehydrogenase-2 (ADH2), and the serotonin autoreceptor (HTR1B) (Blum et al., JAMA, 263:2055, 1990; Goldman and Bergen, Arch Gen Psychiatry, 55:964, 1998; and Lappalainen et al., Arch Gen Psychiatry, 55:989, 1998). However, as in most psychiatric disorders the mode of inheritance is complex and multifactorial, and undoubtedly influenced by environment (Devor and Cloninger, Annu Rev Genet, 23:19-36, 1989).

Thus, there remains a need in the art for the identification of additional genes, which play a role in alcohol abuse and/or dependence. In particular, the molecular definition of polymorphisms at alcohol abuse and/or alcohol dependence loci will prove useful for accurate diagnosis of various alcoholism subtypes. Identification of alcoholism susceptibility alleles is also contemplated to provide tools for the development of new medications to help individuals remain sober.

### SUMMARY OF THE INVENTION

The present invention relates to compositions and methods for determining whether an individual is predisposed to or protected from alcoholism. In particular, the present invention provides genetic markers useful for the diagnosis, characterization, and treatment or prevention of alcohol tolerance, abuse and/or dependence.

In particular the present invention provides methods of identifying individuals predisposed to alcohol tolerance, abuse and/or dependence comprising: providing a sample (e.g., cells) from a human subject; and screening the sample for a biomarker selected from the group consisting of Grid2, Efnb3, Grin1, Zfp179, Tceb11, Gria1, Sec8, Prdx5, Rad50, Catna2, and B2m, wherein the biomarker is correlated with a predisposition to alcohol tolerance and dependence. In some preferred embodiments, the biomarker is selected from the group consisting of Grid2, Efnb3, Grin1, Zfp179, Tceb11, Gria1, and Sec8, and the biomarker is associated with transcriptional upregulation. In other preferred embodiments, the biomarker is selected from the group consisting of Prdx5, Rad50, Catna2, and B2m, and the biomarker is associated with transcriptional downregulation. Also provided are embodiments, wherein the screening is accomplished by quantitating the mRNA or protein level of the biomarker. In some preferred embodiments, the mRNA level is quantitated by a method selected from the group consisting of real time polymerase chain reaction, and gene expression array. In other embodiments, the screening is accomplished by detection of a polymorphism in the biomarker (e.g., coding region of gene, transcription control element, etc.). In particularly preferred embodiments, the detection is accomplished using at least one technique selected from the group consisting of polymerase chain reaction, heteroduplex analysis, single stand conformational polymorphism analysis, ligase chain reaction, comparative genome hybridisation, Southern blotting and sequencing.

Moreover, the present invention provides methods of identifying individuals at reduced risk of developing alcohol dependence, comprising: providing a sample (e.g., DNA) from a human subject; and screening the sample for at least one biomarker of the 16p13.3 locus or the 16q12.2 locus, wherein the biomarker is correlated with a reduced risk of developing at least one symptom of alcohol dependence and/or reduced alcohol consumption. In preferred embodiments, the biomarker is selected from the group consisting of the AC9 gene, the AC7 gene, and markers in linkage disequilibrium thereof, and wherein the screening is accomplished by detection of a polymorphism in the biomarker. In some embodiments, the biomarker is the AC9 gene, and the polymorphism is a [TAA]₉ repeat in an intron of the AC9 gene. In other embodiments, the biomarker is the AC7 gene, and the polymorphism is an [AACA]₇ repeat in the 3′-untranslated region of the AC7 gene. In related embodiments, the at least one symptom comprises a symptom selected from the group consisting of alcohol tolerance (e.g., a need for markedly increased amounts of alcohol to achieve intoxication or desired effect, or markedly diminished effect with continued use of the same amount of alcohol), alcohol withdrawal (e.g., withdrawal syndrome, or use of alcohol or related substance to relieve or avoid withdrawal symptoms), heavy alcohol consumption, persistent desire to reduce alcohol consumption, considerable time spent in acquiring, using or recovering from alcohol, alcohol-related reduction in social, occupational, or recreational activities, and continued alcohol use despite alcohol-related physical (e.g., continued drinking despite recognition that an ulcer was made worse by alcohol consumption) or psychological problems In preferred embodiments, the detection is accomplished using at least one technique selected from the group consisting of polymerase chain reaction, heteroduplex analysis, single stand conformational polymorphism analysis, ligase chain reaction, comparative genome hybridisation, Southern blotting and sequencing.

Furthermore, the present invention provides methods of identifying individuals at increased risk for alcohol abuse, comprising: providing a sample (e.g., DNA) from a human subject; and screening the sample for at least one biomarker of the 16q12.2 locus, wherein the biomarker is correlated with increased risk of developing at least one symptom of alcohol abuse. In some embodiments, the biomarker is selected from the group consisting of the AC7 gene and a marker in linkage disequilibrium with the AC7 gene, and the screening is accomplished by detection of a polymorphism in the marker. In preferred embodiments, the biomarker is the AC7 gene, and the polymorphism is an [AACA]₅ repeat in the 3′-untranslated region of the AC7 gene. In related embodiments, the at least one symptom comprises a symptom selected from the group consisting of recurrent alcohol use resulting in failure to fulfill major obligations, recurrent alcohol use in physically hazardous situations, recurrent alcohol-related legal problems, and continued alcohol use despite alcohol-related social problems. In preferred embodiments, the detection is accomplished using at least one technique selected from the group consisting of polymerase chain reaction, heteroduplex analysis, single stand conformational polymorphism analysis, ligase chain reaction, comparative genome hybridisation, Southern blotting and sequencing.

The present invention also provides kits for determining whether a subject is at a reduced or increased risk for developing alcohol abuse and/or dependence, comprising: at least one reagent capable of specifically detecting at least one polymorphism in an allele of a gene selected from the group consisting of AC9, AC7, and genes in linkage disequilibrium thereof; and instructions for determining whether a subject is at a reduced risk of developing alcohol abuse and/or dependence. In preferred embodiments, the at least one polymorphism is a microsatellite repeat polymorphism. The present invention provides embodiments wherein the at least one reagent comprises a nucleic acid probe that hybridizes under stringent conditions to a nucleic acid sequence selected from the group consisting of the coding strand of the gene, and the noncoding strand of the gene. In some preferred embodiments, the at least one reagent comprises a sense primer and an antisense primer flanking the at least one polymorphism in the allele. In related embodiments, at least one of the primers comprises a fluorescent tag.

Also provided by the present invention are methods of screening compounds, comprising: providing: i) at least one allele of a gene selected from the group consisting of Grid2, Efnb3, Grin1, Zfp179, Tceb11, Gria1, Sec8, Prdx5, Rad50, Catna2, B2m, AC9, and AC7, or a cell transfected with the at least one allele of the gene, and ii) one or more test compounds; and contacting the gene or the cell with the test compound; and detecting a change in expression of the gene in the presence of the test compound relative to the absence of the test compound. In some embodiments, the detecting comprises detecting RNA transcribed from the gene, while in other embodiments the detecting comprises detecting a polypeptide encoded by the gene. Additionally, the present invention provides embodiments wherein the gene is contained within an eukaryotic cell when contacting the gene with the test compound. In other embodiments, the gene is contained within an in vitro transcription reaction when contacting the gene with the test compound.

### DESCRIPTION OF THE FIGURES

FIG. 1 depicts the results obtained from testing HAFT (H) and LAFT (L) mice for acute functional tolerance (AFT) to the incoordinating effect of ethanol in panel A, and to blood ethanol concentration at which balance is regained for a second time (BEC2) in panel B. Asterisks denotes p<0.05 by t-test with 3-5 mice per group.

FIG. 2 provides a diagram of the number (corners of the square) of differentially expressed transcripts (same direction in two replicate lines) determined by two statistical procedures (t-test noise distribution or permutation, using the 80th percentile cutoff) when data are initially analyzed using MAS v.4.0 or MAS v.5.0, as indicated at the corners of the square. The common differentially expressed transcripts are indicated with boxed numbers adjacent to the connecting lines.

FIG. 3 provides graphs indicating the quantity of N-methyl-D-aspartate receptor 1 (NR-1) subunit (panel A) and zinc finger 179 (panel B) mRNA detected by real time-PCR using whole brain total RNA from HAFT-1 and LAFT-1 mice (generation 24). The mRNA levels shown were based on a standard curve and normalized to 18S rRNA.

FIG. 4 shows that the differential expression of N-methyl-D-aspartate receptor 1 (NR1) subunit and zinc finger 179 (Zfp 179) mRNA between HAFT and LAFT mice was quantitatively similar when measured by microarrays and by real time PCR.

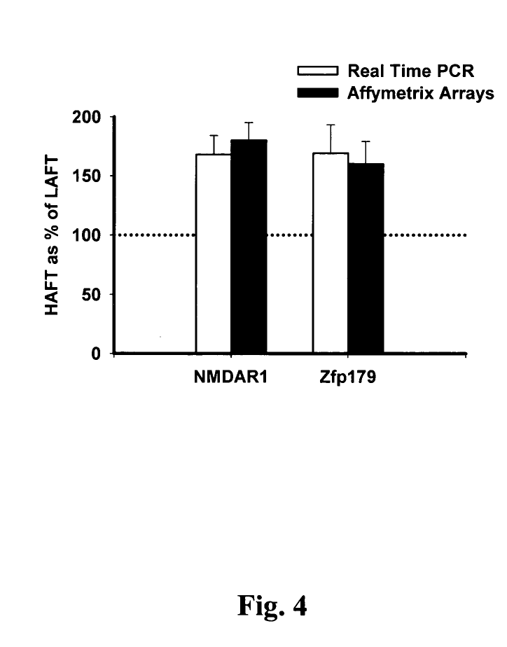

FIG. 5 provides a schematic of the putative pathways mediating acute functional (physiologic) tolerance to the incoordinating effect of ethanol.

FIG. 6 provides the results of ROC analysis demonstrating the predictive power of the presence of the AC9.R9 allele with respect to alcohol consumption in males (panel A), and in females (panel B), respectively. For males, the cutoff was 692 g alcohol/30 days, χ² was 11.6 (p=0.001), and the OR was 2.35, while for females, the cutoff was 1154 g alcohol/30 days, χ² was 13.67 (p<0.0001), and the OR was 4.7. The cutoffs were determined by ROC analysis.

### GENERAL DESCRIPTION OF THE INVENTION

Acute functional tolerance to ethanol develops during a single exposure to ethanol, and has been suggested to be a predisposing factor for alcohol dependence. Genetic components to acute functional tolerance, as well as to alcohol dependence, have been clearly demonstrated. By using a combination of selective breeding, quantitative trait locus (QTL) analysis, and DNA microarray technology, candidate genes for the complex phenotype of ethanol tolerance have been identified. In particular, a signal transduction cascade involving the GluRδ2 protein, the Ephrin B3 ligand, the NMDA receptor and tyrosine kinase activity, has been identified as a modulator or mediator of acute tolerance to the incoordinating effect of ethanol in a murine model.

Case-control association studies for alcohol abuse, alcohol consumption and alcohol dependence were performed. For this purpose, polymorphic markers in the human 16p13.3 locus and in the human 16q12.2 locus were used. The first marker used was a trinucleotide repeat located in the second intron of the adenyl cyclase type IX (AC9) gene. Logistic regression analyses of association between multiple subject characteristics and the AC9.R9 allele were performed. The results of these analyses indicated that alcohol consumption was significantly, negatively, associated with the AC9.R9 allele. The second marker used was the D16S475 marker, which was selected as it had previously been shown to be associated with an alcohol severity phenotype (Foroud et al., Alcohol Clin Exp Res, 22:2035-2042, 1998). Using chi-square analysis of association between multiple subject characteristics and the D16S475.R11 allele, a negative association was found between alcohol dependence and the D16S475.R11 allele. However, upon further more sophisticated statistical analysis, this relationship could not be confirmed. The third marker used was a tetranucleotide repeat located in the 3′-untranslated region of the adenylyl cyclase type VII (AC7) gene. Multivariate analyses between multiple subject characteristics and the AC7.R7 and AC7.R5 alleles were performed. The results of these analyses indicated that alcohol dependence was significantly negatively associated with the AC7.R7 allele (e.g., the presence of this allele was associated with a decreased likelihood of or protection from alcohol dependence). In contrast, the presence of the AC7.R5 allele was associated positively with alcohol abuse (e.g., the presence of the AC7.R5 allele was associated with an increased likelihood of or predisposition to alcohol abuse).

Definitions

To facilitate understanding of the invention, a number of terms are defined below.

The terms “subject” as used herein, refers to a human. It is intended that the term encompasses healthy individuals, as well as, individuals predisposed to, or suspected of having a predisposition toward alcoholism. Typically, the terms “subject” and “patient” are used interchangeably. In some preferred embodiments of the present invention, the term subject refers to specific subgroups of patients including but not limited to Caucasians, females, and alcohol-dependent individuals. As used herein, the term “Caucasian” refers to a member of the white race consisting of individuals of European, north African, or southwest Asian ancestry. The term “female” encompasses both women and girls.

As used herein, the term “alcohol tolerance” refers to a phenomenon wherein a greater amount of alcohol needs to be consumed to obtain the same effect or less effect is evident if a tolerant individual consumes the same amount of alcohol as he/she did previously (DSM-IV). As used herein, the term “alcohol abuse” refers to a behavior of an individual in relation to alcohol consumption as defined by DSM III-R or DSM-IV criteria. As used herein, the term “alcohol-dependent” refers to a behavior of an individual addicted to alcohol and diagnosed as such by DSM III-R or DSM-IV criteria.

As used herein, the terms “adenylyl cyclase” and “adenylate cyclase” refer to a class of enzymes responsible for the biosynthesis of cAMP from ATP. In preferred embodiments, the terms “adenylyl cyclase 9” and “AC9” refer to human adenylyl cyclase type IX, while the terms “adenylyl cyclase 7” and “AC7” refer to human adenylyl cyclase type VII.

The term “gene” refers to a nucleic acid (e.g., DNA) sequence that comprises coding sequences necessary for the production of a polypeptide (e.g., AC9), precursor, or RNA (e.g., mRNA). The polypeptide can be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid” refers to any nucleic acid containing molecule, including but not limited to, DNA, cDNA and RNA. In particular, the terms “AC9 gene” and “AC9 nucleic acid” refer to the full-length AC9 nucleotide sequence. The terms “AC9 gene” and “AC9 nucleic acid” as used herein, also encompass fragments of the AC9 sequence, as well as other domains within the full-length AC9 nucleotide sequence. Furthermore the term “AC9 nucleotide sequence” encompasses DNA, cDNA, and RNA. In some preferred embodiments, the AC9 nucleic acid refers to a portion of an AC9 intron (e.g., SEQ ID NO:1). In particularly preferred embodiments, the AC9 intron is intron 2. In other embodiments, the term AC9 nucleic acid refers to the AC9 mRNA (e.g., GenBank Accession No. NM_{—}00116) or to the AC9 gene (e.g., portion of Homo sapiens chromosome 16 contig of GenBank Accession No. NT_{—}010552). Additionally, the terms “AC7 gene” and “AC7 nucleic acid” refer to the full-length AC7 nucleotide sequence. The terms “AC7 gene” and “AC7 nucleic acid” as used herein, also encompass fragments of the AC7 sequence, as well as other domains within the full-length AC7 nucleotide sequence. Furthermore the term “AC7 nucleotide sequence” encompasses DNA, cDNA, and RNA. In some preferred embodiments, the AC7 nucleic acid refers to a portion of the 3′-untranslated region of AC7 (e.g., SEQ ID NO:15). In other embodiments, the term AC7 nucleic acid refers to the AC7 mRNA (e.g., GenBank Accession No. NM_{—}001114) or to the AC7 gene (e.g., portion of Homo sapiens chromosome 16 contig of GenBank Accession No. NT_{—}010505).

In addition to containing introns, genomic forms of a gene may also include sequences located on both the 5′ and 3′ end of the coding sequences that are present on the RNA transcript. These sequences are referred to as “flanking” sequences or regions. The 5′ flanking region may contain regulatory sequences such as promoters and enhancers that control or influence the transcription of the gene. The 3′ flanking region may contain sequences that influence the transcription termination, post-transcriptional cleavage, mRNA stability and polyadenylation.

As used herein, the term “portion of a chromosome” refers to a discrete section of the chromosome. Human chromosomes are divided into sites or sections by cytogeneticists as follows: the short (relative to the centromere) arm of a chromosome is termed the “p” arm; the long arm is termed the “q” arm. Each arm is then divided into two regions termed region 1 and region 2. Region 1 is closest to the centromere. Each region is further divided into bands. The bands may be further divided into sub-bands. For example, the 16p13.3 portion of human chromosome 16 is the portion located on the short arm (p) in the first region (1) in the 3rd band (3) in sub-band 3 (0.3).

The term “portion” used in reference to a nucleotide sequence (as in “a portion of a given nucleotide sequence”) refers to fragments of that sequence. The fragments may range in size from 12 nucleotides to the entire nucleotide sequence minus one nucleotide. In some embodiments, the term portion refers to nucleic acid fragments of at least 24 nucleotides in length. In preferred embodiments, the fragments are at least 48 nucleotides in length, in particularly preferred embodiments, the fragments are at least 96 nucleotides in length. In some embodiments, nucleic acid fragments of at least 12 nucleotides in length are suitable for use as probes or primers.

As used herein, the term “biomarker” refers to DNA, RNA or protein that is correlated with a particular condition. In some preferred embodiments, the biomarker refers to a DNA, RNA or protein that is correlated with a predisposition to developing alcohol tolerance and/or dependence. In some of these embodiments the biomarker comprises either a greater or lesser level of mRNA transcribed from a gene of interest, or a greater or lesser level of protein encoded by a gene of interest. In some other preferred embodiments, the biomarker comprises a polymorphism in a DNA, RNA and/or protein. In particularly preferred embodiments, the biomarker comprises both a polymorphism, as well as altered mRNA or protein levels. Preferred biomarkers are selected from the group consisting of: Grid2, Efnb3, Grin1, Zfp179, Tceb11, Gria1, Sec8, Prdx5, Rad50, Catna2, B2m, AC9, and AC7. However, the present invention is not limited to this list of biomarkers. In fact additional suitable biomarkers are detected using the methods and compositions described in the experimental examples.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “polymorphism” refers to the regular and simultaneous occurrence in a single interbreeding population of two or more alleles of a gene, where the frequency of the rarer alleles is greater than can be explained by recurrent mutation alone (typically greater than 1%). In some preferred embodiments, the term “polymorphism” refers to a trinucleotide repeat polymorphism in an intron of AC9. In some particularly preferred embodiments, the trinucleotide repeat polymorphism is [TAA]₉ in the second intron of AC9. In other preferred embodiments, the term “polymorphism” refers to a tetranucleotide repeat polymorphism in the 3′-untranslated region of AC7. In particularly preferred embodiments, the tetranucleotide repeat polymorphism is selected from [AACA]₇ and [AACA]₅.

The term “microsatellite repeat” as used herein refers to a variety of simple di-(dinucleotide repeats such as [GA]₁₁), tri-(trinucleotide repeats such as [TAA]₉), tetra-(tetranucleotide repeats such as [AACA]₅), and pentanucleotide tandem repeats that are dispersed in the euchromatic arms of most chromosomes.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide protein) chain. The DNA sequence thus codes for the amino acid sequence.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

The term “Northern blot” as used herein refers to the analysis of RNA by electrophoresis of RNA on agarose gels, to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps; the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acids. Amplification techniques have been designed primarily for this sorting out.

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “sense primer” refers to an oligonucleotide capable of hybridizing to the noncoding strand of gene. The term “antisense primer” refers to an oligonucleotide capable of hybridizing to the coding strand of a gene. In some embodiments, when used in reference to the biomarkers for which the term coding and noncoding strand may not be relevant, the term “sense primer” refers to the primer designated herein as the “forward primer” and thus the strand to which this primer hybridizes is equivalent to that of a noncoding strand. Conversely, in some embodiments, the term “antisense primer” refers to the primer designated herein as the “reverse primer” and thus the strand to which this primer hybridizes is equivalent to that of the coding strand.

As used herein, the term “fluorescent tag” refers to a molecule having the ability to emit light of a certain wavelength when activated by light of another wavelength. “Fluorescent tags” suitable for use with the present invention include but are not limited to fluorescein, rhodamine, Texas red, TET, HEX, TAMRA, ROX, JOE, Cy5, Cy3, and Oregon Green.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “real time PCR” as used herein, refers to various PCR applications in which amplification is measured during as opposed to after completion of the reaction. Reagents suitable for use in real time PCR embodiments of the present invention include but are not limited to TaqMan™ probes, molecular beacons, Scorpions™ primers or double-stranded DNA binding dyes.

As used herein the terms “TaqMan™” and “5′nuclease assay” refer to a PCR-based assay, which makes use of the inherent 5′ to 3′ exonuclease activity of the Thermus aquaticus (Taq) DNA polymerase to simultaneously generate a target-specific signal and a target-specific amplification product (Holland et al., Proc Natl Acad Sci USA, 88:7276-7280, 1991). Specificity is achieved through the use of three target-specific oligonucleotides: including a forward primer, a reverse primer, and a non-extendable probe (e.g., TaqMan™ probe) capable of hybridizing between the two primers (e.g., downstream). In fluorogenic TaqMan™ assays, the probe contains a reporter dye at its 5′ end and a quencher dye at its 3′ end. If the target sequence is present in the sample, during the extension step Taq DNA polymerase will cleave the hybridized probe thereby causing the reporter and quencher dyes to be separated resulting in a detectable increase in fluorescence. In the absence of the target sequence, the probe remains intact, permitting quenching of the reporter dye.

Real-time monitoring of PCR reactions can also be accomplished with three target specific oligonucleotides without depending upon the 5′ exonuclease activity of the DNA polymerase to generate a detectable signal. In one case, an internal probe or a “molecular beacon” is used which emits a fluorescent signal upon hybridization to the target DNA (Tyagi and Kramer, Nat. Biotechnol., 14:303-308, 1996). Like fluorogenic TaqMan™ probes, molecular beacons also contain a reporter dye (e.g., 5-(2′-aminoethyl) aminonaphtahlene-1-sulfonic acid, EDANS) at their 5′ terminus and a quencher dye (e.g., 4-(4′-dimethylaminophenylazo)benzoic acid, DABCYL) at their 3′ terminus. However, in contrast to TaqMan™ probes, molecular beacons contain self-complementary sequences at both termini, which are capable of forming a stem loop structure thereby bringing the two dyes in close proximity. In the presence of a target sequence, a signal is generated during the annealing phase of a PCR reaction as binding of the molecular beacon to the target DNA serves to separate the reporter and quencher dyes.

The term “Amplifluors™” as used herein refers to molecular beacon variants which combine both the signal generating (e.g., 6-fluorescein reporter and DABCYL quencher) and primer functions in a single oligonucleotide (Nazarenko et al., Nucleic Acids Res, 25:2516-2521, 1997). To reduce PCR artifacts, “Scorpions™-type Amplifluors™” containing a loop sequence, which is complementary to the amplicon can be used, to insure that a signal is generated only upon amplification of the desired target sequence (Whitcombe et al., Nat Biotechnol, 17:804-807, 1999).

In addition, “dsDNA-specific dyes” such as SYBR Green I, are viable alternatives to the use of sequence-specific probes for real-time PCR (Wittwer et al., BioTechniques, 22:130-138, 1997). In solution, the unbound dye exhibits very little fluorescence, however, upon dsDNA-binding during the extension phase, fluorescence is greatly enhanced. This technique requires the optimization of reaction conditions, in order to minimize deleterious background fluorescence caused by dye binding to non-specific amplification products (e.g., primer-dimers).

The term “transcriptional upregulation” as used herein refers to an increase in synthesis of RNA, by RNA polymerases using a DNA template. For example, when used in reference to the methods of the present invention, the term “transcriptional upregulation” refers to an increase of least 2 fold, more preferably 2 fold to 3 fold, yet more preferably 3 to 10 fold, and most preferably greater than 10 fold, in the quantity of mRNA corresponding to a gene of interest detected in a sample derived from an individual predisposed to alcohol tolerance, abuse or dependence as compared to that detected in a sample derived from an individual who is not predisposed to alcohol tolerance, abuse or dependence. In particularly preferred embodiments, the increase is statistically significant.

Conversely, the term “transcriptional downregulation” refers to a decrease in synthesis of RNA, by RNA polymerases using a DNA template. For example, when used in reference to the methods of the present invention, the term “transcriptional downregulation” refers to a decrease of least 2 fold, more preferably 2 fold to 3 fold, yet more preferably 3 to 10 fold, and most preferably greater than 10 fold, in the quantity of mRNA corresponding to a gene of interest detected in a sample derived from an individual predisposed to alcohol tolerance, abuse or dependence as compared to that detected in a sample derived from an individual who is not predisposed to alcohol tolerance, abuse or dependence. In particularly preferred embodiments, the decrease is statistically significant.

Both transcriptional “upregulation” and transcriptional “downregulation” may also be indirectly monitored through measurement of the translation product or protein level corresponding to the gene of interest. The present invention is not limited to any given mechanism related to upregulation or downregulation of transcription.

The terms “array,” “chip,” “probe array,” and “microarray” refer to a small solid surface (e.g., glass) on which thousands of oligonucleotide or polynucleotide probes have been deposited (e.g., robotically) and immobilized in a predetermined order permitting automated recording of sample hybridization information. Some embodiments of the present invention comprise “GeneChip® expression arrays” (Affymetrix) for the qualitative and quantitative measurement of gene expression levels in a biologically relevant organism (e.g., human, rat, mouse, etc.).

The term “eukaryotic cell” as used herein refers to a cell or organism with membrane-bound, structurally discrete nucleus and other well-developed subcellular compartments. Eukaryotes include all organisms except viruses, bacteria, and bluegreen algae.

As used herein, the term “in vitro transcription” refers to a transcription reaction comprising a purified DNA template containing a promoter, ribonucleotide triphosphates, a buffer system that includes DTT and magnesium ions, and an appropriate RNA polymerase, which is performed outside of a living cell or organism.

The term “amplification reagents” as used herein, refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “ligase chain reaction” and “ligase amplification reaction” refer to methods for detecting small quantities of a target DNA, with utility similar to PCR. Ligase chain reaction relies on DNA ligase to join adjacent synthetic oligonucleotides after they have bound the target DNA. Their small size means that they are destabilized by single base mismatches and so form a sensitive test for the presence of mutations in the target sequence.

The terms “single-strand conformation polymorphism” and “SSCP,” as used herein, refer to the ability of single strands of nucleic acid to take on characteristic conformations under non-denaturing conditions, which in turn can influence the electrophoretic mobility of the single-stranded nucleic acids. Changes in the sequence of a given fragment (i.e., mutations) will also change the conformation, consequently altering the mobility and allowing this to be used as an assay for sequence variations (Orita et al., Genomics 5:874-879, 1989).

As used herein, the terms “conformation-sensitive gel electrophoresis” or “CSGE” refer to methods for detecting mutations, which involve distinguishing DNA heteroduplexes from homoduplexes via mildly denaturing gel electrophoresis. CSGE protocols are well known in the art (Ganguly et al., Proc Natl Acad Sci USA 90:10325-10329, 1993).

The term “DNA sequencing” refers to methods used to determine the order of nucleotide bases in a DNA molecule or fragment. The term “DNA sequencing” includes for example, dideoxy sequencing and Maxam-Gilbert sequencing.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

The terms “test compound” and “candidate compound” refer to any chemical entity, pharmaceutical, drug, and the like that is a candidate for use to treat or prevent a disease, illness (e.g., alcohol tolerance, abuse or dependence), sickness, or disorder of bodily function. Test compounds comprise both known and potential therapeutic compounds. A test compound can be determined to be therapeutic by screening using the screening methods of the present invention.

As used herein, the term “sample” is meant to include a specimen obtained from a subject. The term “sample” encompasses fluids, solids, and tissues. In preferred embodiments, the term “sample” refers to blood or biopsy material obtained from a living body via any appropriate technique (e.g., needle, sponge, scalpel, swab, etc.), for the purpose of examination.

The term “alcohol tolerance” as used herein refers to a phenomenon wherein a greater amount of alcohol needs to be consumed to obtain the same effect or less effect is evident if a tolerant individual consumes the same amount of alcohol as he/she did previously (DSM-IV).

The term “alcohol abuse” as used herein refers to a clinical syndrome (See, DSM-IV) that includes one or more of the following over 1 year: alcohol use despite social or interpersonal problems; alcohol use in physically hazardous situations; alcohol use resulting in failure to fulfill obligations; recurrent alcohol-related fights; and alcohol-related legal problems.

The term “alcohol dependence” as used herein refers to a clinical syndrome (See, DSM-IV) that includes at least three of the following over 1 year: tolerance (e.g., increased drinking to achieve same effect); alcohol withdrawal signs; drinking more alcohol than intended; unsuccessful attempts to cut down on use; excessive time related to alcohol (e.g., obtaining, hangover); impaired social or work activities due to alcohol; and use despite physical or psychological consequences. While alcohol tolerance is a phenomenon that is evident in both alcohol abusing and alcohol dependent individuals, the phenomenon of alcohol abuse is differentiated from alcohol dependence by the DSM-IV criteria for these phenomena (e.g., alcohol abusing individuals are ones that meet the criteria for abuse, but do not meet the criteria for dependence).

The terms “antisocial personality disorder” and “ASPD” as used herein refer to a clinical syndrome (See, DSM-IV) that includes: a pervasive pattern of disregard for and violation of the rights of others as indicated by at least three of: failure to conform to social norms regarding lawful behavior by repeatedly performing acts that are grounds for arrest; deceitfulness, as indicated by repeated lying, use of aliases, or conning others; impulsivity or failure to plan ahead; irritability and aggressiveness, as indicated by repeated physical fights or assaults; reckless disregard for the safety of self or others; consistent irresponsibility, as indicated by repeated failure to sustain consistent work behavior or failure to honor financial obligations; lack of remorse, as indicated by being indifferent to or rationalizing having hurt, mistreated, or stolen from another. In addition individuals with ASPD are at least 18 years of age, there is evidence of conduct disorder with onset before age 15, and the occurrence of antisocial behavior is not exclusively during a schizophrenic or manic episode. Physical violence is currently a prerequisite.

As used herein, the term “risk of developing alcohol abuse or dependence” refers to a subject's relative risk (e.g., the percent chance or a relative score) of developing alcohol abuse or dependence during their lifetime.

The term “subject suspected of being alcohol dependent” refers to a subject that presents one or more symptoms indicative of alcohol dependence (e.g., physiologic tolerance, withdrawal symptoms, excessive use, etc.) or is being screened for alcohol dependence (e.g., during a routine physical).

As used herein, the term “alcohol-related” refers to phenomena associated with alcohol consumption. For example, the phrase “alcohol-related legal problems,” refer to legal problems (e.g., drunk driving/driving under influence/driving while intoxicated: the crime of operating a motor vehicle while under the influence of alcohol) associated with alcohol intake.

As used herein, the term “diagnosis” refers to the determination of the nature of a case of disease. In some preferred embodiments of the present invention, methods for making a diagnosis are provided which permit determination of susceptibility to develop alcohol tolerance, abuse or dependence.

The term “reagent(s) capable of specifically detecting a microsatellite polymorphism” refers to reagents used to detect a repeat polymorphism from a gene of interest, a cDNA, or a RNA. Examples of suitable reagents include but are not limited to nucleic acid probes capable of specifically hybridizing to genomic DNA.

As used herein, the term “instructions for determining whether a subject is predisposed to alcohol dependence” refers to instructions for using the reagents contained in the kit for the detection and characterization of an alcohol dependence susceptibility allele (e.g., AC7.R5) in a sample from a subject. In contrast, the term “instructions for determining whether a subject is protected from alcohol dependence” refers to instructions for using the reagents contained in the kit for the detection and characterization of an alcohol dependence resistance allele (e.g., D16S475.R11, AC9.R9, AC7.R7, etc.) in a sample from a subject. In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination. Additional information is available at the Internet web page of the U.S. FDA.

### DESCRIPTION OF THE INVENTION

The present invention relates to compositions and methods for determining whether an individual is predisposed to or protected from alcohol tolerance, abuse and/or dependence. In particular, the present invention provides genetic markers useful for the diagnosis, characterization and treatment of alcoholism. In the first place, the present invention encompasses compositions and methods involving genes whose transcriptional upregulation or downregulation is associated with a predisposition to acute functional tolerance to ethanol and to alcohol dependence. Specifically, elevated levels of Grid2, Efnb3, Grin1, Zfp179 and Tceb11 transcripts, and reduced levels of Prdx5 transcripts, were found to be associated with alcohol tolerance in selected lines of mice, and the homologs of these genes are expected to be associated with alcohol tolerance in humans. In the second place, the present invention encompasses compositions and methods involving the human AC9.R9 allele or the D16S475.R11 allele and protection from alcohol dependence. Specifically, the D16S475.R11 allele is associated with protection from an alcoholism subtype characterized by a compulsion to drink, while the AC9.R9 allele is associated with protection from an alcoholism subtype characterized by drink-induced aggression. In addition, the present invention encompasses alleles in linkage disequilibrium with the AC9.R9 allele and protection from high alcohol consumption and/or dependence. Furthermore, the present invention encompasses the human AC7.R7 and AC7.R5 alleles, and alleles in linkage disequilibrium with either the AC7.R7 allele or the AC7.R5 allele, and protection from alcohol abuse and/or dependence, respectively.

I. Alcohol Tolerance in a Murine Model

Tolerance to alcohol (i.e., ethanol) is an important component of the DSM-IV and ICD-10 diagnoses of alcohol dependence (See, Diagnostic and Statistical Manual of Mental Disorders, 4th ed., American Psychiatric Association, Washington, D.C., 1994; and The ICD-10 Classification of Mental and Behavioral Disorders, Diagnostic Criteria for Research, World Health Organization, Geneva, 1993). Tolerance that develops during a single exposure to alcohol is defined as acute functional tolerance (AFT) or within-session tolerance, and has been considered as a predisposing element for alcohol dependence (Newlin and Thomson, Psychol Bull, 108:383-402, 1990). A genetic component to acute functional alcohol tolerance has clearly been demonstrated by the bidirectional selective breeding of lines of mice to display high acute functional tolerance (HAFT mice) and low acute functional tolerance (LAFT mice) to the incoordinating effect of ethanol (Erwin and Deitrich, J Pharmacol Exp Ther, 279:1310-1317, 1996). The technique of selective breeding enhances the representation or frequency of genetic material associated with a particular trait, which shifts the animal's phenotype away from the population mean (See, Falconer and Mackay, Introduction to Quantitative Genetics, Longman, Essex, 1996). Therefore, HAFT mice are expected to have more genetic material that promotes a higher level of acute functional tolerance, while LAFT animals accumulate less of this genetic material and/or more genetic material leading to lower acute functional tolerance.

The chromosomal location of particular genes that influence acute functional ethanol tolerance has recently been investigated with quantitative trait locus (QTL) analysis (Kirstein et al., J Pharmacol Exp Ther, 302:1238-1245, 2002). This technique is a statistical analysis of the association between a complex phenotype (e.g., degree of acute tolerance expressed in an individual animal) and the occurrence of specific marker alleles in the animal's genome (Crabbe et al., Trends Neurosci, 22:173-179, 1999). Thus, if a particular genetic marker occurs at a statistically higher frequency in an animal with high or low acute functional tolerance, it can be inferred that a gene near or at the maker contributes to the trait. However, each gene or QTL may contribute to only a modest degree to the behavior in question. QTL analyses are often carried out using panels of recombinant inbred strains of animals. In particular, regions of the mouse genome that harbor genes affecting acute functional tolerance, have been identified by performing a QTL analysis with BXD recombinant inbred strains of mice (Kirstein, 2002, supra). However, QTL analysis only identifies relatively large chromosomal regions, not the critical gene or genes involved. The genetic difference underlying the QTL could be a polymorphism in the coding region of the gene, leading to a difference in the functional activity of the gene product, or a polymorphism, which could alter the level of transcript produced, or the stability of the transcript, or all of the above.

During development of the present invention, DNA microarray analysis has been utilized to identify genes that are differentially expressed in the brains of HAFT and LAFT mice. These gene expression experiments are contemplated to provide an indication of the genetic elements contributing to the phenotypic differences between the selected lines via the level of transcription. However, without further information, a definitive role of any individual differentially-expressed gene in contributing to acute functional tolerance cannot be convincingly established (See, Falconer and Mackay, 1996, supra; and Crabbe et al., Alcohol Clin Exp Res, 14:141-151, 1990). On the other hand, if the differentially-expressed genes in selectively-bred animals are located within a QTL for the same phenotypic trait for which the animals are bred, more confidence can be given to the premise that a differentially expressed gene is a predisposing factor for the expression of the selected phenotype. Therefore, the genes that were differentially expressed in the brains of HAFT and LAFT mice were also mapped to the identified QTL regions for acute functional tolerance, yielding a set of genes that are contemplated to contribute to this trait.

Selective breeding has been used extensively to investigate the genetics of alcohol-related behaviors (Crabbe et al., Alcohol Clin Exp Res, 14:141-151, 1990). When the phenotypic difference between selected lines has reached a plateau, it is assumed that the alleles associated with the selected behavior are fixed, while the frequencies of unrelated genes remain variable among individuals of the selected lines. During development of the present invention, a high sensitivity threshold was used to initially estimate differential gene expression in the brains of selected lines of HAFT and LAFT mice, resulting in an identification of more than 300 genes. The identified genes displayed differential expression in the same direction in replicate lines of HAFT and LAFT animals (HAFT1, LAFT1, and HAFT2, LAFT2). Because of the high sensitivity and low specificity of the initial screen, there are contemplated to be false positives among the initially identified, differentially expressed genes. Specificity was subsequently greatly enhanced by adding filters that limited the genes of interest to those in which: 1) the differences had to be statistically significant, and had to be in the same direction in both of the selected lines of mice; 2) the differentially expressed genes had to be localized within QTLs associated with acute functional tolerance; and 3) the differences had to be statistically significant regardless of the initial microarray analysis procedure or statistical method used to detect differences. The use of these techniques resulted in the identification of a small set of genes that are contemplated to play important roles in acute functional ethanol tolerance. The present study is the first to examine the genetic underpinning of a behavioral phenotype using a combination of selective breeding, QTL analysis and gene expression analysis.

There are several important issues that must be considered with respect to the present identification of genes associated with the phenotype of acute functional tolerance. In the first place, many probes on the Affymetrix arrays represent ESTs, rather than known genes. Where possible, Affymetrix software and public databases were used to determine the chromosomal localization of differentially expressed ESTs, and to determine sequence homology to known genes. One differentially-expressed EST localized within a QTL was identified in this manner, but a large percentage of the differentially expressed ESTs could not be precisely localized, and/or were not homologous to known genes. These ESTs may, however, represent potentially important genes that cannot at present be fully appreciated. Secondly, the differentially expressed genes that have been identified herein are localized within some, but not all, of the QTLs that have been determined for acute functional tolerance (Kirstein et al., J Pharmacol Exp Ther, 302:1238-1245, 2002). The current method does not permit the identification of genes that lie within QTLs, and which differ in their coding regions (rather than their transcript levels), such that the function of the gene product may be altered.

However, many of the differentially-expressed genes localized within QTLs were identified regardless of the method for analysis of the array data or the statistical method used to determine differential expression. The restriction that genes had to be differentially expressed in the same direction in both replicate lines of selected HAFT and LAFT animals also provides confidence in the results, since the replicate lines represent completely independent selections for the same phenotype (i.e., different populations of heterogeneous stock mice were used to start the two selective breeding experiments). It is also important to note that the QTL analysis was carried out using the identical behavioral test that was used for selection (Erwin et al., J Pharmacol Exp Ther, 279:1310-1317, 1996; and Kirstein et al., 2002, supra). Thus, the differentially expressed genes that are localized within the QTLs are contemplated to be involved in acute functional tolerance to the incoordinating effect of ethanol. In this respect, it may be important to note that the HAFT and LAFT lines also differ in the development of tolerance to the incoordinating effect of ethanol as measured on a rotarod apparatus (Rustay et al., Pharmacol Biochem Behav, 70:167-174, 2001; and Deitrich et al., Alcohol Clin Exp Res, 24:595-604, 2000), but do not differ in tolerance to other effects of ethanol (e.g., hypnotic effect; change in body temperature). Therefore, the association of the identified genes cannot be generalized to acute tolerance to all physiological/pharmacological effects of ethanol.

Some of the differentially expressed genes that are localized within the QTLs for AFT can be readily related to the measured behavior, and can be positioned within a signal transduction pathway that includes elements previously associated with neuroadaptation. The GluRδ2 protein is considered to be a glutamate receptor subunit, based on its sequence homology with AMPA and NMDA subtypes of glutamate receptor (Yamazaki et al., FEBS Lett, 300:39-45, 1992). However, no glutamate receptor functions have yet been associated with the GluRδ2 subunit. The GluRδ2 protein is expressed exclusively in cerebellar Purkinje neurons (Araki et al., Biochem Biophys Res Commun, 197:1267-1276, 1993), and its function has been investigated in null mutant mice and in mice that have naturally-occurring mutations of the Grid2 or GluRδ2 gene (Kashiwabuchi et al., Cell, 81:245-252, 1995; Lalouette et al., Neuroscience, 105:443-455, 2001; and Zuo et al., Nature, 388:769-773, 1997). Analysis of null mutant mice revealed that the GluRδ2 protein plays an important role in motor coordination, formation of parallel and climbing fiber-Purkinje cell synapses, and long-term depression of parallel fiber-Purkinje cell synaptic transmission. A number of mutations of Grid2 have been described (e.g., “hotfoot” alleles), and behavioral analysis of mice with two of these mutant alleles revealed deficits reminiscent of behavior observed in LAFT mice. That is, the LAFT mice require more training trials to reach criterion on the stationary dowel (e.g., ability to balance on the dowel for 5 minutes), and the same was observed for mice with the mutant GluRδ2 alleles, compared to wild-type mice (Lalouette et al., 2001, supra). Another mouse with a mutation in the GluRδ2 gene, Lurcher, also displays severe ataxia, apparently as a result of selective death of Purkinje neurons during development (Zuo et al., 1997, supra).

Beyond the effect of GluRδ2 on motor coordination, however, is the possible role of this protein (and the products of other genes identified in this study), in signal transduction pathways associated with both learning and acute tolerance to ethanol. For example, the response to ethanol in fyn null mutant mice has been examined (Miyakawa et al., Science, 278:698-701, 1997). Fyn is a non-receptor tyrosine kinase of the Src family that mediates tyrosine phosphorylation of NMDA receptors (Nakazawa et al., J Biol. Chem., 276:693-699, 2001). In wild-type mice, the ethanol-induced inhibition of NMDA receptor-mediated hippocampal field potentials was reversed during the time that ethanol was still present in the slice preparation indicating that acute functional tolerance to ethanol's inhibitory effect on NMDA receptor function had developed (Miyakawa et al., 1997, supra). This acute tolerance was not observed in the fyn knockout mice, suggesting that the lack of acute tolerance was related to the demonstrated inability of ethanol to increase tyrosine phosphorylation of the NMDA receptor in the fyn null animals. The GluRδ2 protein forms complexes with scaffolding proteins such as delphilin and PSD-93 (Miyagi et al., J Neurosci, 22:803-814, 2002; and Roche et al., J Neurosci, 19:3926-3934, 1999), that anchor it to actin and to signaling molecules, such as the Src tyrosine kinases located at the parallel fiber-Purkinje cell synapse. Furthermore, the GluRδ2 protein has been reported to interact directly with a protein tyrosine phosphatase, PTPMEG (Hironaka et al., J Biol Chem, 275:16167-16173, 2000). Activation of the PTPMEG tyrosine phosphatase increased Fyn-mediated tyrosine phosphorylation of the NMDA receptor subunit, NR2A (Hironaka et al., 2000, supra). Therefore, a lower level of GluRδ2 in LAFT mice is contemplated to be associated with decreased Fyn phosphorylation of the NMDA receptor.

Importantly, ephrin B3 (an ephrin ligand) as well as GluRδ2 was expressed at lower levels in the brains of LAFT, as compared to HAFT mice. Ephrin receptors are receptor tyrosine kinases that are activated by ligands, including ephrin B3 (Kullander and Klein, Nat Rev Mol Cell Biol, 3:475-486, 2002). Ephrin (Eph) receptors and ephrins are both membrane-bound proteins generally considered to be localized on apposing cells. Clustering of ligands and receptors by PDZ proteins is necessary for Eph receptor signaling, which may be bi-directional. That is, activation of Eph receptors by ephrins results in association of adaptor molecules with the receptor, which leads to intracellular signaling. However, interaction of ephrins with the Eph receptors also results in “reverse signaling” via tyrosine phosphorylation of the cytoplasmic regions of the ephrins (Kullander and Klein, 2002, supra). Interestingly, both ephrins and Eph receptors can interact with Fyn. In particular, it has been shown that Eph receptor activation by certain members of the ephrin ligand family activates the NMDA receptor, and that following ephrin activation, NR2B is tyrosine phosphorylated on the same residues that are phosphorylated by Fyn (Murai and Pasquale, Neuron, 33:159-162, 2002). If ephrin B3 plays a similar role as other ephrins, then the lower levels of ephrin B3, like the lower levels of GluRδ2, in brains of LAFT mice, are contemplated to result in decreased tyrosine phosphorylation of NMDA receptors and decreased acute functional ethanol tolerance.

Activation of EphB receptors also stimulates an interaction between NR1 and the Eph receptors, and results in co-clustering of EphB receptors and NR1 (Dalva et al., Cell, 103:945-956, 2000). Notably, NR1 expression is reduced in brains of LAFT mice, compared to HAFT mice, and the combination of lower ephrin B3 and lower NR1 may therefore lead to a lower number, and less surface-expressed, NMDA receptors. Such a phenomenon is contemplated to further exacerbate the contribution of decreased tyrosine phosphorylation of NMDA receptors to acute functional ethanol tolerance.

The transcript for peroxiredoxin 5 is also differentially expressed, being higher in LAFT mice than in HAFT mice, and localized within a QTL for AFT. Peroxiredoxins are antioxidant proteins that reduce hydrogen peroxide and organic hydroperoxides using electrons donated by thioredoxin, glutathione, and other molecules (Chae et al., Proc Natl Acad Sci, USA 91:7017-7021, 1994). These enzymes have been suggested to be important in antioxidant defense and in redox signaling. NMDA receptor activation can increase levels of nitric oxide and hydroxyl radicals (Cambonie et al., J Neurosci Res, 62:84-90, 2000; and Marin et al., Eur J Neurosci, 4:425-432, 1992). Hydrogen peroxide and hydroxyl radicals can activate MAP kinases (Crossthwaite et al., J Neurochem, 80:24-35, 2002), and NMDA receptor activation also stimulates the MAP kinase pathways (Platenik et al., Life Sci, 67:335-364, 2000). If the higher level of peroxiredoxin reduces the steady-state and induced levels of hydrogen peroxide, then even the downstream function of the NMDA receptor is contemplated to be reduced in brains of LAFT mice.

The combination of differentially expressed genes identified herein, provides a picture of disrupted modulation of the NMDA receptor function in brains of LAFT mice. In particular, tyrosine phosphorylation of the NMDA receptor, which has been implicated in acute functional tolerance to alcohol, is contemplated to be impaired in LAFT mice. Given the exclusive localization of GluRδ2 in cerebellar Purkinje cells, it is important to note that NMDA receptor subunits have also been localized to adult Purkinje cells (Thompson et al., Neurosci Lett, 283:85-88, 2000). In addition, there is an electrophysiological NMDA response in Purkinje cells, although its characteristics are different from responses observed in other neurons (Miyahi et al., J Neurosci, 22:803-814, 2002; and Misra et al., J Physiol, 525:299-305, 2000). Thus, impaired NMDA receptor phosphorylation/function in the cerebellum or other brain regions of LAFT mice is contemplated to be a contributing factor in the reduced acute functional tolerance to the incoordinating effect of ethanol in LAFT mice. It is worth noting, that the selection of HAFT and LAFT mice was asymmetric in that AFT decreased during selection in LAFT mice to a greater degree than AFT increased in HAFT mice (Erwin and Deitrich, J Pharmacol Exp Ther, 279:1310-1317, 1996). Therefore, the differential gene expression observed in the present studies is contemplated to represent a change during selection in the LAFT mice, while gene expression in HAFT mice may be similar to that of the heterogeneous stock animals from which the lines were selected.

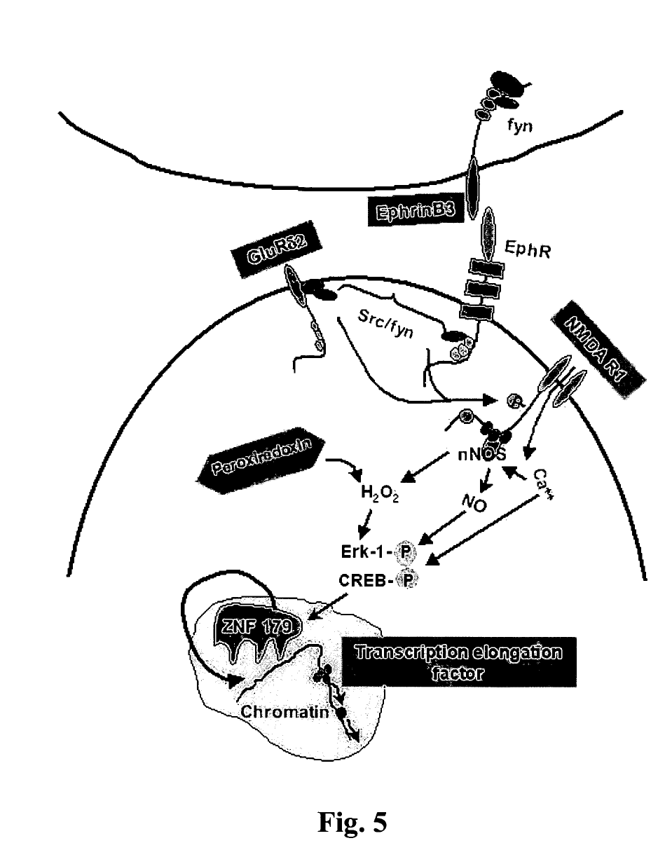

The other genes that showed consistent differences in expression, and that were localized within QTLs for AFT, were the zinc finger protein 179 and a transcription elongation factor. Interestingly, the zinc finger protein 179 is a transcription factor and a member of the RING finger family, which can be induced rapidly by activation of NMDA receptors (Zhao et al., Genomics, 49:394-400, 1998; and Ohkawa et al., J Neurochem, 78:75-87, 2001). Zinc finger protein 179 acts in concert with other factors to control transcriptional events. The efficacy of transcriptional machinery is supported by transcription elongation factors (Reines et al., Curr Opin Cell Biol, 11:342-346, 1999; and Sowden et al., Genomics, 29:145-151, 1995), which in turn are suggested to be involved in the generation of specific neuronal types during development (Guo et al., Nature, 408:366-369, 2000), as well as in DNA repair and recombination. The reduced expression of transcription factor genes in brains of LAFT mice is likely to influence the expression of other specific genes that may, in a secondary fashion, contribute to acute functional ethanol tolerance. On the other hand, the lower NMDA receptor function in brains of LAFT mice may be directly linked to lower expression of the zinc finger protein 179. A schematic illustration of pathways linking the differentially expressed genes identified herein is shown in FIG. 5.

The differential expression of genes in whole brains of HAFT and LAFT mice were analyzed because, a priori there was no basis for choosing a brain area to be associated with acute functional tolerance. The selective expression of certain genes of interest (e.g., GluRδ2) in the cerebellum now provides some evidence, that a particular brain area may be functionally important to development of acute functional tolerance to the incoordinating effect of ethanol.

In some instances, the differentially expressed genes within QTLs for acute functional tolerance are contemplated to reflect polymorphisms in regulatory regions of the genes that affect transcription factor binding. As an initial exploration of this possibility, bioinformatic techniques were used to assess putative transcription factor binding sites associated with the differentially expressed genes. As shown in Table 5, consensus binding sites for members of the CCAAT/enhancer binding protein (C/EBPα) family, which have intrinsic and indirect cAMP-inducible activity (Wilson et al., Mol Cell Endocrinol, 181:27-34, 2001), as well as constitutive activity (Wilson and Roesler, Mol Cell Endocrinol, 188:15-20, 2002), were found in all of the genes for which promoter information could be generated, and that were consistently differentially expressed and localized in QTLs for AFT. Consensus sequences for binding of other transcription factors could also be identified. In general, transcription factors bind to specific DNA sequences and increase or decrease transcription of particular genes by interactions with the RNA polymerase transcription complex. In reality, this interaction is more complex, and includes recruitment of coactivator complexes, as well as sequential activation of promoters and interactions among transcription factors.

Clinical studies by Schuckit and colleagues have indicated that individuals with a positive family history of alcohol dependence (FHP) show blunted physiological, behavioral (including incoordination), and subjective responses to alcohol (low response to alcohol), compared to those with a negative family history of alcohol dependence (FHN; Schuckit and Smith, J Stud Alcohol, 61:827-835, 2000). It has been suggested that this blunted response may reflect greater levels of acute functional tolerance in the FHP individuals (Newlin and Thomson, Psychol Bull, 108:383-402, 1990). In particular, the low alcohol responders (primarily FHP) have been found to have a significantly greater risk of becoming alcohol dependent (Schuckit and Smith, 2000, supra). More recently, four chromosomal regions in humans have been identified that were correlated with a low level of response to alcohol (Schuckit et al., Alcohol Clin Exp Res, 25:323-329, 2001). Given that this low response level might be associated with acute functional tolerance in humans, the previously identified human chromosomal regions were compared to the QTL regions for AFT in mice, in order to determine whether the differentially expressed genes identified herein may be homologs of genes associated with AFT/low response to ethanol in humans. By examination of syntenic regions of mouse and human genomes, two mouse QTLs for AFT (chromosomal locations: 11:29-43 and 1:81.1-89.2) were found to be syntenic with regions of the human genome that had been identified as QTLs for low response to ethanol (Schuckit et al., 2001, supra). Of the four genes located in the mouse QTLs, two (transcription elongation factor and peroxiredoxin 5) were located within or near the human QTL regions. Whether the identified mouse genes within these QTLs also show different expression between humans with a high and low response to ethanol remains to be determined. However, this combination of results provides strong suggestive evidence that a common set of genes is important for rapid adaptation to ethanol in humans, as well as in mice. Considering that the altered response to ethanol in humans is predictive of the development of alcohol dependence, analysis of the identified gene expression differences in humans using the methods disclosed herein, is contemplated to generate specific information regarding genetic influences on human alcohol dependence.

II. Alcohol Dependence in a Human Population

The heritable nature of alcoholism was first shown more than three decades ago, and is still one of the most replicated and enduring findings in the study of psychiatric genetics (Goodwin et al., Arch Gen Psychiatry, 28:238-243, 1973; Cotton, Journal of Studies on Alcohol, 40:89-116, 1979; Cloninger et al., Arch Gen Psychiatry, 38:861-868, 1981). It has also become clear that alcoholism is a heterogeneous trait under the influence of multiple genetic and environmental factors (Tabakoff and Hoffman, Pub Health Report, 103:690-698, 1988; Cloninger, Science, 236:410-416, 1987). During the development of the present invention, the sequence of the AC9 gene located at 16p13.3 was examined. A trinucleotide repeat located in the second intron of the AC9 gene was found to be highly polymorphic and was used herein for the purpose of performing an association analysis between alcohol dependence, alcohol consumption, and the AC9 gene locus. Additionally, a tetranucleotide repeat located in the 3′-untranslated region of the AC7 gene, located at 16q12.2, was found to be polymorphic and was used herein for the purpose of performing an association analysis between alcohol dependence, alcohol abuse, and the AC7 gene locus. The study subjects were also genotyped for the D16S475 dinucleotide polymorphism previously used by the Collaborative Study on the Genetics of Alcoholism group to define their alcohol severity phenotype (Foroud et al., Alcohol Clin Exp Res, 22:2035-2042, 1998). The D16S475 polymorphism was chosen in part to serve as a positive control for the association study between the AC9 intron polymorphism and alcohol dependence. The value of using two markers in close proximity (2 cM) to one another as a positive control depends on the assumption that the markers will exhibit linkage disequilibrium with each other. Linkage disequilibrium depends on a variety of factors in addition to the cytogenic distance between two markers. For example, regional variability in recombination patterns, age of mutation, growth rate within a population, ethnic diversity, and mating patterns within a population can all significantly impact the level of linkage disequilibrium (Cardon and Bell, Nature Reviews, 2:91-98, 2001).

In an initial analysis described in greater detail in the experimental examples 7-10 below, specific alleles from two microsatellite markers, one in an intron of the AC9 gene (AC9.R9), and the other located within 2 cM upstream of the AC9 gene (D16S475.R11) are each significantly associated with a protective influence on an individual's predisposition to alcohol dependence. The Cochran-Armitage Trend Test was used to screen for potential association between Major Depression (MD), Alcohol Dependence (AD) and Antisocial Personality Disorder (ASPD) and the two polymorphisms located at the 16p13.3 locus. The results of the Cochran-Armitage Trend Test indicated that only the AC9.R9 (nine repeat) allele and the D16S475.R11 (eleven repeat) allele showed any trend for association with the three psychiatric screening variables. Moreover, both the AC9.R9 (χ²=8.9, p=0.003) and the D16S475.R11 (χ²=12, p=0.001) alleles were significantly associated with a protective influence on alcohol dependence (See, Table 6). In contrast the AC9.R9 and D16S475.R11 alleles were not associated with major depression, and only marginally associated with antisocial personality disorder. Interestingly, the marginal association between the D16S475.R11 and AC9.R9 alleles, and ASPD was protective, as was the case for alcohol dependence.

The finding that two polymorphisms in or near the AC9 locus are associated with alcohol dependence indicates that a variation in the AC9 gene influences an individual's liability for alcohol dependence. Accordingly, an individual's AC9 genotype is a suitable trait-marker for alcohol dependence. However, several factors needed to be considered. First, results shown in Table 7 (AC9.R9) and Table 9 (D16S475.R11) indicate that study site and ethnicity are potentially confounding factors in the assessment of an association between each marker and alcohol dependence. For this reason, multiple logistic regression analysis was used to further examine the effects of the AC9.R9 and the D16S475.R11 alleles on alcohol dependence, after adjusting for ethnicity and study site variables (See, Table 8 and Table 10). Logistic regression is ideally suited for analysis of the combined effect of multiple factors on binary outcomes (diagnoses). The odds ratio values provided as part of the logistic regression output can be interpreted as indicative of the increased or decreased disease risk that is associated with any factor in the logistic regression model, after simultaneously adjusting for the influence of each of the other factors in the model. For example, results from the logistic regression analysis shown in Table 8 indicate that in addition to the AC9.R9 allele (p=0.004, OR=0.52, 95% CI=0.34-0.81), ethnicity (p=0.00003) and ASPD (p=0.000001, OR=12.4, 95% CI=6.8-22.6) are significantly related to alcohol dependence (Table 8, Model 1). In other words, the AC9.R9 genotype remains a significant risk factor even after adjusting for the effects of ethnicity and ASPD. To further address the possibility of population stratification resulting in confounding of the relationship between the AC9.R9 allele and alcohol dependence, another logistic regression analysis was performed on a more ethnically homogeneous (e.g., white or Caucasian) subgroup (Table 8, Model 2). After including only the Caucasian subjects in the logistic regression, the significance of the effect of the AC9.R9 allele on alcohol dependence was unchanged (p=0.002, OR=0.51, 95% CI=0.33-0.79). The consistency of the odds ratio and significance values when comparing a model that included only white subjects to a model that included black, white, and Asian subjects, suggests that the relationship between the AC9.R9 allele and alcohol dependence is valid, and not simply due to population stratification. In addition, a chi-square analysis in which only Caucasian subjects from a single study site (Montreal) were included showed that the significance of the association between the AC9.R9 allele and alcohol dependence was maintained (p=0.0001, OR=0.43, 95% CI=0.26-0.71). This result suggests that the relationship between the AC9.R9 allele and alcohol dependence is not an artifact of the study site.

Logistic regression was also used to examine the association between the D16S475.R11 allele and alcohol dependence, as well as the potentially confounding effects of study site, ethnicity, and/or ASPD. The results of the logistic regression analyses shown in Table 10 (Model 1), indicate that in addition to the D16S475.R11 allele (p=0.006, OR=0.6, 95% CI=0.42-0.86), ethnicity (p=0.001) and ASPD (p=0.000001, OR=10.7, 95% CI=5.3-20.5) were significantly associated with alcohol dependence. Interpreted another way, the results of Table 10 (Model 1) indicate that after adjusting for the effects of ethnicity and ASPD, the D16S457.R11 allele remains significantly associated with alcohol dependence. To further address the possibility that population stratification was confounding the relationship between the D16S475.R11 allele and alcohol dependence, logistic regression analysis was again performed on a more ethnically homogeneous (e.g., white or Caucasian) subgroup (Table 10, Model 2). In agreement with the results from the analysis of the AC9.R9 allele, there was no change in the significance or odds ratio for the association between the D16S475.R11 allele and alcohol dependence. This finding clearly indicates that the relationship between the D16S475.R11 allele and alcohol dependence is real, and not simply an artifact of population stratification.

The extremely high level of correlation between ASPD and alcohol dependence in the study disclosed herein, is suggestive of an “endophenotype” for alcoholism. In particular, the ASPD symptomology, in addition to the standard DSM-IV diagnostic criteria for alcohol dependence, could lead to a more definitive phenotype. Indeed, an earlier report described a sub-group of alcoholics (Type II) that were characterized by a high level of antisocial personality traits (Cloninger, Science, 236:410-416, 1987). The possibility that the Type I and Type II alcoholics could be distinguished by a difference in stimulated platelet adenyl cyclase (AC) activity was then investigated (Parsian et al., Alcoholism: Clinical and Experimental Research, 20:745-751, 1996). These experiments showed a significantly lower fluoride stimulated AC activity in alcoholic subjects, but did not find a difference in fluoride-stimulated AC activity between the two types of alcoholics indicating that low AC activity in alcoholics may be related to characteristics not distinguished by the type I and type II criteria. During development of the present invention, forskolin-stimulated platelet AC activity was also unable to distinguish between alcohol dependent and non-alcohol dependent subjects with or without ASPD. This finding is consistent with a more recent work showing that AC9 protein lacks the forskolin activation domain found in all other known isoforms of AC (Hacker et al., Genomics, 50:97-104, 1998). Although ASPD was shown to be significantly associated with alcohol dependence (See, Table 8 and Table 10), only a marginal association between ASPD and the AC9.R9 and D16S475.R11 alleles was observed (See, Table 7 and Table 9). Thus even though ASPD is related to alcohol dependence, it is unclear whether the association is due to genetic factors or whether ASPD is simply a closely correlated phenotype or a manifestation of the heavy alcohol consumption that accompanies alcohol dependence.

Since both the AC9.R9 allele and the D16S475.R11 allele were each individually, negatively associated with alcohol dependence, two possibilities exist. Either each marker is in linkage disequilibrium with a common functional mutation that influences liability for alcohol dependence, or the two markers are in linkage disequilibrium with separate functional gene mutations both of which influence an individual's liability for alcohol dependence. Since the distance between the D16S475 marker and the AC9 marker is approximately 2 cM (2,000 kb), the second possibility could be expanded to include either two separate disease-causing mutations at a single locus (AC9) or could be due to mutations in two different genes, each of which influences an individual's liability for alcohol dependence, independent of one another. To further test the effect of both alleles as association analysis was performed. As shown in Table 11, the subjects were grouped according to whether they exhibited one, both or neither of the alleles. Also, for the purpose of minimizing potential population stratification, the analysis for association between alcohol dependence and the four allele combinations was performed after selection of a relatively homogeneous group of subjects from the Montreal Study Site. The results of this analysis showed an overall highly significant association between the marker pairs and alcohol dependence (χ²=²², p=0.00006). In addition, logistic regression was used to look for an interaction between the AC9.R9 and the D16S475.R11 alleles. As shown in Table 12, the results from pair-wise consideration of the AC9.R9 and D16S475.R11 alleles indicates that the D16S475.R11 and AC9.R9 alleles are in linkage disequilibrium with separate mutations either in a single gene, or within different genes each of which influences an individual's risk for alcohol dependence. The latter situation is contemplated to be more likely because although the logistic regression for alcohol dependence including each of the alleles (D16S475.R11 and AC9.R9) showed significant main effects from each allele, logistic regression did not show a significant interaction between the D16S475.R11 and AC9.R9 alleles. One interpretation of these findings is that the AC9.R9 allele and the D16S475.R11 allele are associated with different “endophenotypes” contained within the broader diagnosis of alcohol dependence. The WHO/ISBRA survey allowed us to compare the AC9.R9 and the D16S475.R11 alleles with regard to the actual symptomology that was used to determine the DSM-UV alcohol dependence diagnosis. The comparison shown in Table 13 indicates that indeed there is substantial difference in the symptom “profile” of those individuals with the D16S475.R11 allele compared to subjects with the AC9.R9 allele. Thus, the two markers are contemplated to be associated with different subgroups of alcohol dependent subjects.

Furthermore, an univariate analysis (Table 14) indicated that males from Helsinki, Finland were 3.4 times more likely to have the AC9.R9 allele if they drank less than 15 g of alcohol (one standard drink)/day and females from Montreal, Canada were 3 times more likely to have the AC9.R9 allele if they drank less than 15 g of alcohol/day. The data for males from Montreal followed the same trend, but the association was not statistically significant.

Multiple logistic regression analysis was then used to explore the odds of having the AC9.R9 allele and consuming lesser quantities of alcohol. For this analysis, only Caucasian subjects were used (to avoid the influence of ethnicity). The variables used for the logistic regression analysis are provided in Table 15. A number of variables were removed prior to the final model building process. Logistic analysis using alcohol consumption as the dependent outcome variable was performed on combined data from Caucasian subjects (males and/or females from Helsinki; Montreal; Sao Paulo, Brazil; and Sydney, Australia). A statistically significant negative (O.R. =0.605) association was evident between the AC9.R9 allele and consumption of more than 15 g of alcohol/day (χ²=5.06, p<0.025). The negative association means that individuals with the AC9.R9 allele consumed less alcohol. However, this association was not observed for the D16S475.R11 allele.

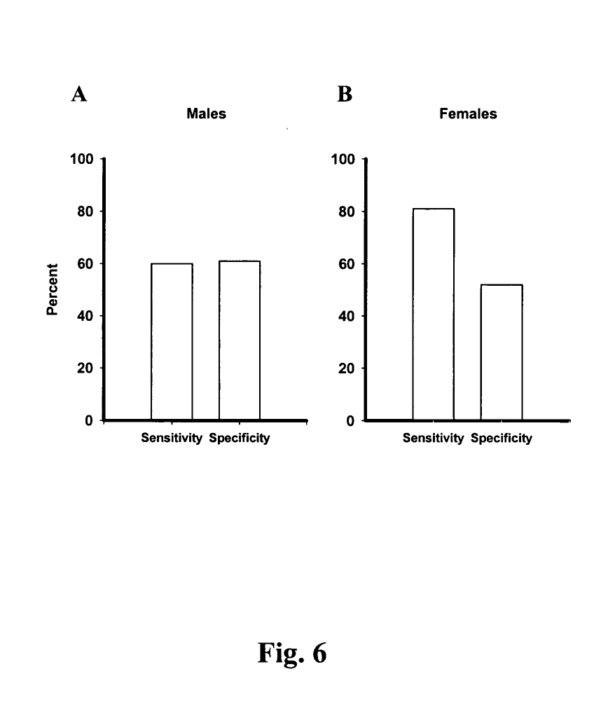

The predictive power of the presence of the AC9.R9 allele with respect to amount of alcohol consumed was determined by receiver-operated characteristic (ROC) analysis (FIG. 6). Using cutoff values for alcohol consumption determined from the ROC analysis, the specificity and sensitivity of the presence of the AC9.R9 allele for predicting alcohol consumption was determined. In the total population of males, specificity was 61% and sensitivity was 60%. In the total population of females, specificity was 52% and sensitivity was 81%. The statistically significant χ² values (FIG. 6) indicate that a smaller proportion of subjects with the AC9.R9 allele will drink an amount of alcohol greater than the cutoff value, and the odds ratio (O.R.) indicates the likelihood that an individual with the AC9.R9 allele will drink an amount of alcohol less than the cutoff value (FIG. 6). The results confirm that the AC9.R9 allele is associated with low alcohol consumption, which in turn is associated with a lower risk for alcohol dependence.

Multiple logistic regression analysis was also used to investigate the odds of having the AC7.R7 or AC7.R5 allele and a particular alcohol-related phenotype. Combined data from Caucasian subjects from Helsinki, Montreal, Sao Paulo and Sydney were used for the AC7.R7 analysis. In this total population, a statistically significant negative association was found between the presence of the AC7.R7 allele and DSM-IV lifetime alcohol dependence. Specifically, the presence of the AC7.R7 allele was associated with a decreased likelihood (odds ratio=0.75) of alcohol dependence (χ²=4.28, p<0.039). This association was statistically significant in males (χ²=3.93, p<0.048), but not in females (p=0.73).

In populations in which alcohol abuse was definitively distinguished from alcohol dependence (combined data from the Montreal population and the population recruited in Bethesda, Md., USA), logistic analysis revealed a significant positive association of the AC7.R5 allele with alcohol abuse (OR=1.97, χ²=12.22, p<0.0005).

Therefore, the presence of particular polymorphic alleles of the AC7 gene can have either a predisposing (AC7.R5) or protective (AC7.R7) effect with respect to alcohol abuse and/or dependence.

III. Detection of Polymorphisms Associated with Predisposition to or Protection from Alcoholism

A. Alleles Associated with Predisposition to Alcohol Tolerance

In some embodiments, the present invention includes alleles of genes that increase a subject's susceptibility to alcohol tolerance. Specifically, alleles of the human homologues of the GluRδ2 receptor, ephrin B3 ligand, NMDAR1, peroxiredoxin 5, zinc finger protein 179, and transcription elongation factor, possessed by individuals who experience acute functional tolerance to the incoordinating effects of alcohol, are contemplated to be genetic markers associated with a predisposition to alcohol dependence. Alleles of any of these genes found in individuals with a family history of alcohol dependence are contemplated to differ from alleles of individuals without such a family history, by inclusion of polymorphisms resulting in altered transcriptional regulation. One or more these polymorphisms are expected to be present in the noncoding regions of the gene, including but not limited to the 5′ regulatory region (e.g., promoter or enhancer binding sites), the 3′-untranslated region (e.g., mRNA stability), or the intron(s) (e.g., RNA splicing), but may also be found in the coding region of the gene. In fact, any polymorphism in the human counterparts of the murine Grid2, Efnb3, Grin1, Prdx5, Zfp179 and Tceb11 genes, which are associated with alcohol tolerance are within the scope of the present invention. For example, in some embodiments, the present invention provides single-nucleotide polymorphisms of the GluRδ2 receptor, while in other embodiments, repeat polymorphisms are provided.

B. Alleles Associated with Protection from High Levels of Alcohol Consumption and/or Alcohol Dependence

In other embodiments, the present invention includes polymorphisms in and around the human 16p13.3 and 16q12.2 loci that decrease a subject's susceptibility to alcohol dependence (e.g., including, but not limited to AC9.R9, AC7.R7 and D16S475.R11). Analysis of naturally occurring human polymorphisms at the 16p13.3 locus revealed that subjects who appear to be protected from alcohol high levels of alcohol consumption and alcohol dependence have a nine trinucleotide repeat in an intron of the AC9 gene (e.g., [TAA]₉ in the second intron of AC9 disclosed herein as SEQ ID NO:2), and subjects protected from alcohol dependence have a higher frequency of an eleven dinucleotide repeat in the D16S475 marker (e.g., [GA]₁₁, disclosed herein as SEQ ID NO:4), in addition to the [TAA]₉ repeat. Analysis of naturally occurring human polymorphisms at the 16q12.2 locus revealed that male subjects who appear to be protected from alcohol dependence have a seven tetranucleotide repeat in the 3′-untranslated region of the AC7 gene (e.g., [AACA]₇ disclosed herein as SEQ ID NO:16). However, the present invention is not limited to the nine trinucleotide repeat polymorphism, the eleven dinucleotide repeat polymorphism, or the seven tetranucleotide repeat polymorphism. In fact, any 16p13.3 or 16q12.2 locus polymorphism associated with predisposition to or protection from high levels of alcohol consumption and/or dependence is within the scope of the present invention. For example, in some embodiments the present invention provides single-nucleotide polymorphisms of AC9 or AC7, while in other embodiments repeat polymorphisms in and around the 16p13.3 or 16q12.2 loci are provided.

C. Alleles Associated with Predisposition to Alcohol Abuse

In other embodiments, the present invention includes polymorphisms in and around the human 16q12.2 locus that increase a subject's susceptibility to alcohol abuse (e.g., including but not limited to AC7.R5). Analysis of naturally occurring human polymorphisms at the 16q12.2 locus revealed that subjects who are diagnosed as alcohol abusers (DSM-IIIR or DSM-IV) have a 5 tetranucleotide repeat in the 3′-untranslated region of the AC7 gene (e.g., [AACA]₅ disclosed herein as SEQ ID NO:17). However, the present invention is not limited to the 5 tetranucleotide repeat polymorphism. In fact, any 16q12.2 locus polymorphism associated with susceptibility to alcohol abuse is within the scope of the present invention. For example, in some embodiments of the present invention, single nucleotide polymorphisms in AC7 are provided, while in other embodiments, repeat polymorphisms in and around the 16p12.2 locus are provided.

D. Detection of Alcohol Tolerance-Susceptibility Alleles and Alcohol Dependence-Resistance Alleles

Accordingly, the present invention also provides methods for determining whether a patient has an increased susceptibility or resistance to alcohol tolerance or dependence by genotyping an individual at the alcohol tolerance and dependence genes disclosed herein. In some embodiments, the present invention provides methods and compositions for identifying at least one allele of a gene selected from the group consisting of the human Grid2, Efnb3, Grin1, Prdx5, Zfp179, Tceb11, AC9, and AC7 genes, and the D16S475 marker. In other embodiments, the present invention provides methods for providing a prognosis of increased or decreased risk for alcohol abuse and/or dependence to an individual based on the presence or absence of one or more mutations in the human Grid2, Efnb3, Grin1, Prdx5, Zfp179, Tceb11, AC9, and AC7 genes, and the D16S475 marker. In some embodiments, the mutation causes alcohol tolerance and/or dependence, while in other embodiments the mutation is in linkage disequilibrium with a mutation causing or contributing to the development of alcohol tolerance and/or dependence.

A number of methods are available for analysis of polymorphisms. Assays for detection of polymorphisms or mutations fall into several categories, including but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention, and are described in relationship to detection of the AC9.R9, AC7.R7 and AC7.R5 alleles. However, the present invention is not limited to detection of this allele or to alleles of the AC9 gene. In fact, detection of at least one allele of the human counterparts of the Grid2, Efnb3, Grin1, Prdx5, Zfp179, and Tceb11 genes or at least one allele of the human counterparts of the Gria1, Sec8, Rad50, Catba2, and B2m genes are within the scope of the present invention.

1. Direct Sequencing Assays

In some embodiments of the present invention, polymorphisms are detected using a direct sequencing technique. In these assays, DNA samples are first isolated from a subject using any suitable method. In some embodiments, the region of interest is cloned into a suitable vector and amplified by growth in a host cell (e.g., a bacterium). In other embodiments, DNA in the region of interest is amplified using PCR.

Following amplification, DNA in the region of interest (e.g., the region containing the polymorphism of interest) is sequenced using any suitable method, including but not limited to manual sequencing using radioactive marker nucleotides, or automated sequencing. The results of the sequencing are displayed using any suitable method. The sequence is examined and the presence or absence of a given polymorphism is determined.

2. PCR Assay

In some embodiments of the present invention, polymorphisms are detected using a PCR-based assay. In some embodiments, the PCR assay comprises the use of oligonucleotide primers to amplify an AC9 or AC7 fragment containing the repeat polymorphism of interest. The presence of an additional repeat in the AC9 or AC7 gene results in the generation of a longer PCR fragment which can be detected by gel electrophoresis. For instance, by use of the method described in Example 9, the AC9.R9 allele is detected by the appearance of a 169 bp PCR product, while other AC9 alleles are detected by the appearance of shorter (e.g., 166 bp) or longer (e.g., 172 bp) PCR products, respectively. Similarly, the AC7.R7 allele is detected by the appearance of a 203 bp product, while other AC7 alleles are detected by the appearance of shorter (e.g., AC7.R5 is detected by the presence of a 196 bp product) or longer PCR products.

In other embodiments, the PCR assay comprises the use of oligonucleotide primers that hybridize only to the mutant or wild type allele of AC9 (e.g., to the region of polymorphism). Both sets of primers are used to amplify a sample of DNA. If only the mutant primers result in a PCR product, then the patient has the mutant AC9 allele. If only the wild-type primers result in a PCR product, then the patient has the wild type allele of AC9.

3. Fragment Length Polymorphism Assays

In some embodiments of the present invention, polymorphisms are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.

a. RFLP Assay

In some embodiments of the present invention, polymorphisms are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

b. CFLP Assay

In other embodiments, polymorphisms are detected using a CLEAVASE fragment length polymorphism assay (CFLP; Third Wave Technologies, Madison, Wis.; See e.g., U.S. Pat. No. 5,888,780). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

4. Hybridization Assays

In preferred embodiments of the present invention, polymorphisms are detected by hybridization assay. In a hybridization assay, the presence or absence of a given polymorphism or mutation is determined based on the ability of the DNA from the sample to hybridize to a complementary DNA molecule (e.g., an oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., polymorphism) is detected directly by visualizing a bound probe (e.g., a Northern or Southern assay; See e.g., Ausabel et al. (eds.), Current Protocols in Molecular Biology, John Wiley & Sons, N.Y., 1991). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.

b. Detection of Hybridization Using “DNA Chip” Assays

In some embodiments of the present invention, polymorphisms and/or differences in levels of gene expression (e.g., mRNA) are detected using a DNA chip hybridization assay. In this assay, a series of oligonucleotide probes are affixed to a solid support. The oligonucleotide probes are designed to be unique to a given polymorphism. The DNA sample of interest is contacted with the DNA “chip” and hybridization is detected.

In some embodiments, the DNA chip assay is a GeneChip (Affymetrix, Santa Clara, Calif.; See e.g., U.S. Pat. No. 6,045,996) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. No. 6,068,818). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. No. 6,001,311). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface followed by removal by spinning.

DNA probes unique for the polymorphism of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

C. Enzymatic Detection of Hybridization

In some embodiments of the present invention, genomic profiles are generated using an assay that detects hybridization by enzymatic cleavage of specific structures (INVADER assay, Third Wave Technologies; See e.g., U.S. Pat. No. 6,001,567). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified genomic DNA. The isolated DNA sample is contacted with the first probe specific either for a SNP/mutation or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. No. 5,952,174). In this assay, SNPs are identified using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

5. Mass Spectroscopy Assay

In some embodiments, a MassARRAY system (Sequenom, San Diego, Calif.) is used to detect polymorphisms (See e.g., U.S. Pat. No. 6,043,031). DNA is isolated from blood samples using standard procedures. Next, specific DNA regions containing the polymorphism of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than 0.0001 second, enabling samples to be analyzed in a total of 3-5 second including repetitive data collection. The SpectroTYPER software then calculates, records, compares and reports, the genotypes at the rate of three seconds per sample.

6. Kits for Analyzing Risk of Alcoholism

The present invention also provides kits for determining whether an individual posseses a specific AC9 or AC7 polymorphism. In some embodiments, the kits are useful in determining whether the subject is at risk of heavy alcohol consumption and/or developing alcohol dependence. The diagnostic kits are produced in a variety of ways. In some embodiments, the kits contain at least one reagent for specifically detecting a mutant AC9 or AC7 allele. In preferred embodiments, the kits contain reagents for detecting a TAA repeat polymorphism in the AC9 gene or an AACA repeat in the AC7 gene. In preferred embodiments, the reagents are primers for amplifying the region of DNA containing the repeat polymorphism. In other preferred embodiments, the reagent is a probe that binds to the polymorphic region. In some embodiments, the kit contains instructions for determining whether the subject is at risk for heavy alcohol consumption and/or developing alcohol dependence. In preferred embodiments, the instructions specify that risk for developing alcohol dependence is determined by detecting the presence or absence of a mutant AC9 or AC7 allele in the subject, wherein subjects having an allele containing a [TAA]₉ or an [AACA]₇ repeat have a decreased risk of heavy alcohol consumption and/or developing alcohol dependence. In other preferred embodiments, the instructions specify that risk for alcohol abuse is determined by detecting the presence or absence of a mutant AC7 allele in the subject, wherein subjects having an allele containing an [AACA]₅ repeat have an increased risk of developing alcohol abuse. In some embodiments, the kits include ancillary reagents such as buffering agents, nucleic acid stabilizing reagents, protein stabilizing reagents, and signal producing systems (e.g., fluorescence generating systems). The test kit may be packaged in any suitable manner, typically with the elements in a single container or various containers as necessary along with a sheet of instructions for carrying out the test. In some embodiments, the kits also preferably include a positive control sample.

7. Bioinformatics

In some embodiments, the present invention provides methods of determining an individual's risk of heavy alcohol consumption, and/or developing alcohol dependence based on the presence of one or more mutant alleles of AC9 or AC7. In some embodiments, the analysis of polymorphism data is automated. For example, in some embodiments, the present invention provides a bioinformatics research system comprising a plurality of computers running a mullet-platform object oriented programming language (See e.g., U.S. Pat. No. 6,125,383). In some embodiments, one of the computers stores genetics data (e.g., the risk of becoming alcohol dependent associated with a given polymorphism). In some embodiments, one of the computers stores application programs (e.g., for analyzing transmission disequilibrium data or determining genotype relative risks and population attributable risks). Results are then delivered to the user (e.g., via one of the computers or via the internet).

IV. Other Utilities

A. Drug Screening

The present invention provides methods and compositions for using at least one mutant human gene or gene product selected from the group consisting of Grid2, Efnb3, Grin1, Prdx5, Zfp179, Tceb11, AC9, Gria1, Sec8, Rad50, Catba2, and B2m as a target for screening drugs that can alter the transcription of the gene or the biological activity of the gene product. The present invention contemplates many means of screening compounds. The examples provided below are presented merely to illustrate a range of techniques available. One of ordinary skill in the art will appreciate that many other screening methods can be used.

A technique for drug screening provides high throughput screening for compounds having suitable binding affinity for at least one mutant human gene selected from the group consisting of Grid2, Efnb3, Grin1, Prdx5, Zfp179, Tceb11, AC9, Gria1, Sec8, Rad50, Catba2, and B2m peptides and is described in detail in WO 84/03564, incorporated herein by reference. Briefly, large numbers of different small peptide test compounds are synthesized on a solid substrate, such as plastic pins or some other surface. The peptide test compounds are then reacted with peptides from the gene of interest and washed. Bound peptides from the gene of interest are then detected by methods well known in the art.

The present invention contemplates the use of cell lines transfected with at least one mutant human gene selected from the group consisting of Grid2, Efnb3, Grin1, Prdx5, Zfp179, Tceb11, AC9, Gria1, Sec8, Rad50, Catba2, and B2m for screening compounds for activity, and in particular to high throughput screening of compounds from combinatorial libraries (e.g., libraries containing greater than 10⁴ compounds). The cell lines of the present invention can be used in a variety of screening methods. In some embodiments, the cells can be used in second messenger assays that monitor signal transduction following activation of cell-surface receptors.

In preferred embodiments, the cells can be used in reporter gene assays that monitor cellular responses at the transcription/translation level. In still further embodiments, the cells can be used in cell proliferation assays to monitor the overall growth/no growth response of cells to external stimuli (e.g., ethanol). In second messenger assays, the host cells are preferably transfected as described above with vectors encoding at least one mutant human gene selected from the group consisting of Grid2, Efnb3, Grin1, Prdx5, Zfp179, Tceb11, AC9, Gria1, Sec8, Rad50, Catba2, and B2m. The host cells are then treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the protein or proteins encoded by the vectors. It is also contemplated that at least some of the compounds in the combinatorial library can serve as agonists, antagonists, activators, or inhibitors of protein acting upstream or downstream of the protein encoded by the vector in a signal transduction pathway (See, FIG. 5).

In some embodiments, the second messenger assays measure fluorescent signals from reporter molecules that respond to intracellular changes (e.g., Ca²⁺ concentration, membrane potential, pH, IP₃, cAMP, arachidonic acid release) due to stimulation of membrane receptors and ion channels (e.g., ligand gated ion channels; See Denyer et al., Drug Discov Today, 3:323, 1998; and Gonzales et al., Drug Discov Today, 4:431-39, 1999). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the compound. Responses of the host cells to treatment with the compounds can be detected by methods known in the art, including, but not limited to, fluorescence microscopy, confocal microscopy (e.g., FCS systems), flow cytometry, microfluidic devices, FLIPR systems (See, e.g., Schroeder and Neagle, J Biomol Screening, 1:75, 1996), and plate-reading systems. In some preferred embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The cells are also useful in reporter gene assays. Reporter gene assays involve the use of host cells transfected with vectors encoding a nucleic acid comprising transcriptional control elements of a target gene (i.e., a gene that controls the biological expression and function of a disease target) spliced to a coding sequence for a reporter gene. Therefore, activation of the target gene results in activation of the reporter gene product.

B. Pharmaceutical Compositions

The present invention further provides pharmaceutical compositions which may comprise all or portions of at least one wild type human gene selected from the group consisting of Grid2, Efnb3, Grin1, Prdx5, Zfp179, Tceb11, AC9, Gria1, Sec8, Rad50, Catba2, and B2m, polypeptides encoded by at least one of these genes, or inhibitors/antagonists of the bioactivity of at least one of these gene products. The polynucleotides, polypeptides, or inhibitors of Grid2, Efnb3, Grin1, Prdx5, Zfp179, Tceb1, AC9, Gria1, Sec8, Rad50, Catba2, and B2m are administered alone or in combination with at least one other agent, such as a stabilizing compound, and may be administered in any sterile, biocompatible pharmaceutical carrier, including, but not limited to, saline, buffered saline, dextrose, and water. In preferred embodiments, the nucleotides or amino acids derived from or encoded by the gene of interest are administered to individuals predisposed to or suffering from alcoholism.

The methods of the present invention find use in altering alcohol tolerance and alcohol dependence or altering physiological states associated with these conditions. Peptides can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy. As is well known in the medical arts, dosage for any one patient depends upon many factors, including the patient's size, body surface area, age, the particular compound to be administered, sex, time and route of administration, general health, and interaction with other drugs being concurrently administered.

These pharmaceutical compositions may be formulated and administered systemically or locally. Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of the active ingredient may be that amount that suppresses any portion of the signal transduction cascade shown in FIG. 5. Determination of effective amounts is well within the capability of those skilled in the art, especially in light of the disclosure provided herein.

For any compound used in the method of the invention, the therapeutically effective dose can be estimated initially from cell culture assays. Then, preferably, dosage can be formulated in animal models (particularly murine models) to achieve a desirable circulating concentration range that adjusts Grid2, Efnb3, Grin1, Prdx5, Zfp179, Tceb11, AC9, Gria1, Sec8, Rad50, Catba2, and B2m mRNA or protein levels.

Normal dosage amounts may vary from 0.1 to 100,000 micrograms, up to a total dose of about 1 g, depending upon the route of administration. Guidance as to particular dosages and methods of delivery is provided in the literature. Those skilled in the art will employ different formulations for the gene of interest than for the inhibitors of the gene of interest.

Experimental

The following examples are provided in order to demonstrate and further illustrate certain preferred embodiments and aspects of the present invention and are not to be construed as limiting the scope thereof.

In the experimental disclosure which follows, the following abbreviations apply: AC (adenylyl cyclase); AC9 (AC type IX); AC9.R9 (AC9 allele containing [TAA]₉ repeat polymorphism); AC7 (AC type VII); AC7. R7 (AC7 allele containing [AACA]₇ repeat polymorphism); AC7.R5 (AC7 allele containing [AACA]₅ repeat polymorphism); D16S475.R11 (D16S475 allele containing [GA]₁₁ repeat polymorphism); EST (expressed sequence tag); cM (centimorgan); UTR (untranslated region); eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); 1 or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); cpm (counts per minute); Ci (Curies); FAM (6-carboxyfluorescein); ROX (carboxy-X-rhodamine); TAMRA (6-carboxytetramethylrhodamine); PCR (polymerase chain reaction); ROC (receiver operated characteristics); CI (confidence interval); OR (odds ratio); SE (standard error); sig (significance); AD (alcohol dependence); AA (alcohol abuse); AFT (acute functional tolerance); ASPD (antisocial personality disorder); BEC (blood ethanol concentration); DSM-IV (Diagnostic and Statistical Manual of Mental Disorders-Fourth Edition); ICD-10 (International Statistical Classification of Diseases and Related Health Problems); ISBRA (International Society for Biomedical Research on Alcoholism); MAS (Microarray Suite); MD (major depression); QTL (quantitative trait locus); and WHO (World Health Organization).

Equipment and reagents were obtained from the following sources: ABI (Applied Biosystems Inc., Foster City, Calif.); Affymetrix (Affymetrix Inc., Santa Clara, Calif.); AGTC (Analytical Genetic Testing Center, Inc., Denver, Colo.); Amersham (Amersham Pharmacia Biotec Inc, Piscataway, N.J.); Apple (Apple, Cupertino, Calif.); Invitrogen (Carlsbad, Calif.); Jackson (The Jackson Laboratory, Bar Harbor, Me.); Life Technologies (Invitogen Life Technologies, Carlsbad, Calif.): PE (Perkin-Elmer, Foster City, Calif.) and Pierce (Pierce Biotechnology, Inc., Rockford, Ill.) Promega (Promega Corp., Madison, Wis.); Qiagen (Qiagen, Valencia, Calif.); and UCHSC (University of Colorado Health Sciences Center, Aurora, Calif.).

### EXAMPLE 1

### Measurement of Acute Functional Tolerance (AFT) to Ethanol

The measurement of acute functional tolerance (AFT) to ethanol was performed in mice of generation 22 (HAFT-1/LAFT-1) and generation 20 (HAFT-2, LAFT-2) as previously described (Erwin and Deitrich, J Pharmacol Exp Ther, 279:1310-1317, 1996; and Kirstein et al., J Pharmacol Exp Ther, 302:1238-1245, 2002). Replicate selected lines of HAFT and LAFT mice (HAFT-1/LAFT-1, HAFT-2/LAFT-2) were obtained from the Institute for Behavioral Genetics. Male mice, approximately 10 weeks of age were used for these experiments. To confirm line identification, HAFT and LAFT mice were genotyped, using a commercially available panel of microsatellite markers (ABI PRISM Mouse Mapping Primers V 1.0). Tail (25-50 mg tissue) DNA was isolated using the DNeasy kit (Qiagen), and genotyping was carried out using the ABI Prism 7000 Sequence Detection System.

Mice were trained to balance on a stationary dowel, and were then given an IP injection of 1.75 g/kg ethanol. At the time when a mouse lost balance on the dowel a retro-orbital sinus blood sample was obtained for measurement of the blood ethanol concentration (BEC0). A second blood sample was obtained when the mouse regained balance on the dowel (BEC1). At this time, a second IP injection of 2.0 g/kg ethanol was given. A third blood sample was taken when the mouse again regained balance on the dowel (BEC2). Blood ethanol concentrations were determined by gas chromatography and the difference between BEC2 and BEC1 was used as the measure of AFT. In the QTL analysis (Kirstein et al., 2002, supra), AFT and BEC2 were found to be significantly genetically correlated (r²=0.82) measures.

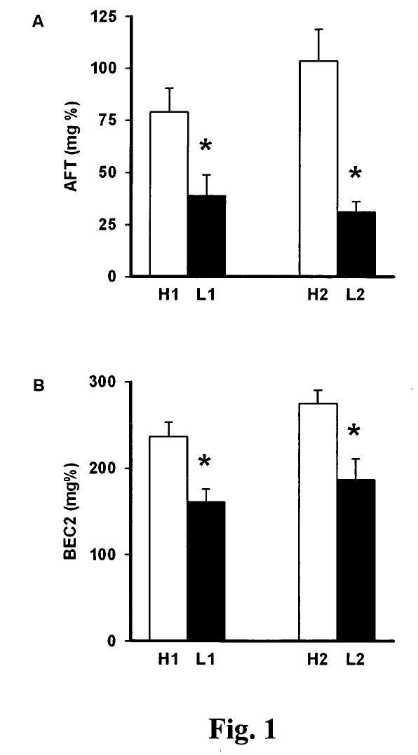

As shown in FIG. 1, HAFT mice of both replicate lines displayed greater acute functional tolerance to the incoordinating effect of ethanol than their LAFT counterparts. The HAFT mice also differed in the highly correlated trait, BEC2 (Kirstein et al., J Pharmacol Exp Ther, 302:1238-1245, 2002).

### EXAMPLE 2

### Gene Expression Analysis

Total RNA was extracted from whole brains of naive HAFT and LAFT mice using the TRIzol reagent (Invitrogen). An additional clean-up of total RNA was carried out using the RNeasy kit (Qiagen).

Affymetrix GeneChip® oligonucleotide arrays (MGU74A arrays versions 1.0 and 2.0; Affymetrix, Santa Clara, Calif.) were used in these experiments. Using the protocol supplied by the manufacturer, double-stranded cDNA was synthesized from total RNA. The cDNA was then used to obtain biotin-labeled cRNA by an in vitro transcription reaction. Biotin-labeled cRNA was fragmented and hybridized with the GeneChip Arrays, according to the manufacturer's protocol, after verifying the quality of the biotin-labeled cRNA on a TestChip. The array was stained with streptavidin-phycoerythrin conjugate and scanned with a Gene Array scanner. RNA from five individual HAFT-1, five LAFT-1, four HAFT-2 and four LAFT-2 mice was hybridized to individual oligonucleotide microarrays.

The image data obtained from the Affymetrix GeneChip® arrays were initially analyzed with two different programs provided by Affymetrix. With each program, a background value is subtracted and a noise correction is applied. A global scaling procedure was used for chip-to-chip normalization. The Microarray Suite (MAS) v.4.0® program then uses an empirical algorithm to determine “present” and “absent” calls and fluorescence intensity levels (average difference values), while Microarray Suite (MAS) v.5.0® uses a statistical algorithm to determine presence or absence and “signal” intensity (Statistical Algorithms Reference Guide, Part No. 701110 Rev. 1, Affymetrix, Inc., Santa Clara, Calif., 2001). MAS v.5.0 includes a detection algorithm that employs probe pair (perfect match/mismatch) intensities to generate a detection p value that is used to determine if a transcript is “present” or “absent”. For the analyses described herein, the default threshold for a “present” call was used. Although, MAS v.4.0 analysis can result in negative average difference calls (meaning that hybridization to mismatch probes is higher than hybridization to perfect match probes), the algorithm used for MAS v.5.0 eliminates this phenomenon. MAS v.5.0 accomplishes this by statistically correcting mismatch values for individual probe pairs within a probe set, where the mismatch value is higher than the perfect match value. Because of the different algorithms used by MAS v.4.0 and MAS v.5.0, different numbers of transcripts can be called “present” or “absent” by the two programs.

In each case, transcripts that were present on all microarrays in a given experiment (e.g., all 5 HAFT-1 and all 5 LAFT-1), or were present on all chips from one line (e.g., HAFT-1) and either absent, marginal, or present on all chips from the other line (e.g., LAFT-1) were subjected to further analysis. Average difference or signal values were subjected to log transformation (negative average difference values in MAS v.4.0 were assigned a value of 1), and two types of statistical analysis were performed to identify genes that were differentially expressed between HAFT and LAFT mice.

The first statistical analysis, t-test Noise Distribution, was similar to a previously published analysis (Eaves et al., Genome Res, 12:232-243, 2002). Differences in gene expression were assessed by calculation of t statistics for each measurement. A filter was then applied that required a minimum threshold value for t. Those thresholds were estimated based on t values for genes with positive values for t in one replicate group (HAFT-1 versus LAFT-1) and negative values for t in the other replicate group (HAFT-2 versus LAFT-2). That is, transcripts that were up-regulated in one replicate group and down-regulated in the other replicate group were used to establish the control distribution of t values for each replicate set, independently of any “real” changes. The value of t for each comparison was required to exceed a given percentile for the control distribution (See Table 1). At each percentile level for the control distribution, the expected number of false positives could be calculated: [(1-percentile)[error rate experiment 1]×(1-percentile) [error rate experiment 2]×(number of genes)×2 [each direction]=number of expected false positives per array-wide comparison]. The expected percentage of false positive values was: 95th percentile, 0.5%; 90th percentile, 2.0%; 85th percentile, 4.5%; 80th percentile, 8% (of the total number of transcripts analyzed).

The second statistical analysis used a permutation procedure, in which the group identities of the samples were randomly permuted, and t values were calculated using the new sample groupings (Dudoit et al., Statistical Methods for Identifying Differentially Expressed Genes in Replicate cDNA Microarray Experiments, Technical Report #578, Stanford School of Medicine, Stanford, Calif., 2000). Using 100 separate permutations of sample group identities, a control distribution was established from the 100×4305 (MAS v.4.0) or 100×3989 (MAS v.5.0) individual t values. This control distribution was then used to determine thresholds at varying percentiles, to filter those genes whose expression level changes exceeded that threshold. The number of expected false positives was calculated as described above.

The t-test Noise Distribution takes advantage of the availability of replicate lines of HAFT and LAFT mice, and assumes that the replicate lines display similar differences in expression of genes that are associated with acute functional tolerance (Crabbe et al., Trends Neurosci, 22:173-179, 1999; and Crabbe et al., Alcohol Clin Exp Res, 14:141-151, 1990). Transcripts that were up-regulated in one comparison (e.g., HAFT-1 vs LAFT-1), and down-regulated in the other comparison (e.g., HAFT-2 vs LAFT-2), were expected to have t-values which were distributed independently of any “real” differences due to selective breeding. Therefore, these t-values could be used to set thresholds such that only a certain number of genes would exceed them by chance (false positives). Data from the Affymetrix arrays were analyzed using Microarray Suite (MAS) v.4.0 or MAS v.5.0, and the total number of transcripts that were analyzed, the number of genes and ESTs found to show statistically significant differences between lines at each threshold, as well as the expected percentage of false positives, are shown in Table 1. Only genes or ESTs that displayed the same direction of differential expression in both replicate lines were considered. A threshold of the 80th percentile was used for this array-wide comparison (the value of t in each comparison had to exceed the 80th percentile of the control distribution). This threshold provided a high sensitivity and therefore avoided exclusion of genes warranting further investigation (false negatives), at the expense of specificity.

TABLE 1
Analysis of RNA from Replicate Lines of HAFT and LAFT Mice

| MAS v.4.0 | MAS v.5.0 |
|---|---|
| (4305) | (3989) |

| | |
|---|---|
| Initial Analysis | # of transcripts with a t |
| (# of transcripts) | value in excess of the |
| Analysis Mode | control distribution¹ |

| | | |
|---|---|---|
| Noise | | |
| Distribution | | |
| Percentile | | |
| 95 | 33 | 26 |
| 90 | 105 | 101 |
| 85 | 231 | 207 |
| 80 | 371 | 395 |
| Permutation² | | |
| Percentile | | |
| 95 | 75 | 71 |
| 90 | 189 | 166 |
| 85 | 287 | 269 |
| 80 | 384 | 353 |

¹Differential expression in same direction in both replicate lines.
²100 permutations.

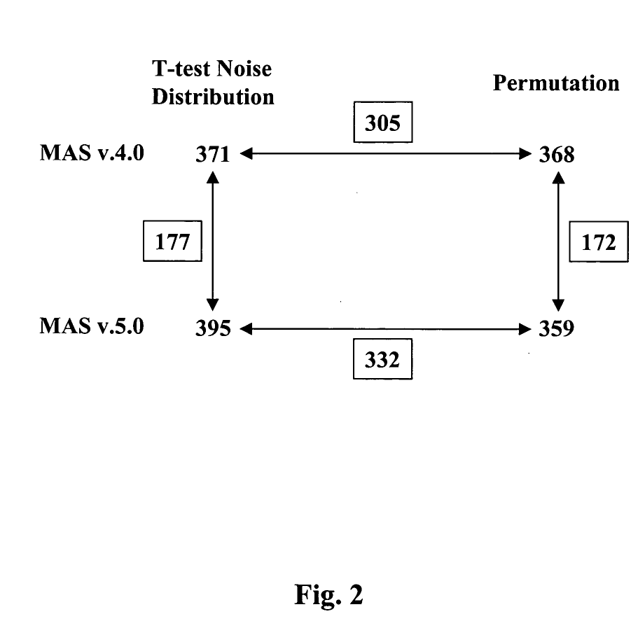

Table 1 also shows the results of the permutation analysis, which is a suitable method for adjusting p values obtained from multiple comparisons (Dudoit et al., Statistical Methods for Identifying Differentially Expressed Genes in Replicate cDNA Microarray Experiments, Technical Report #578, Stanford School of Medicine, Stanford, Calif., 2000). This analysis was also performed on data obtained using MAS v.4.0 or MAS v.5.0. One hundred permutations of these data resulted in a control distribution of t values, which allowed a determination of percentile thresholds for filtering differential gene expression in the replicate lines of mice. Again, only genes with the same direction of differential expression in both replicate lines of mice were considered. For the permutation analysis, a significance threshold at the 80th percentile was again chosen to avoid exclusion of possibly important genes. The procedure used to normalize microarray data has been reported to exert a greater influence on the detection of differentially expressed genes, than does the statistical method used to determine differences (Hoffman et al., Geno Biol, 3:1-11, 2002). To determine the influence of the initial data analysis (MAS v.4.0 versus MAS v.5.0), as compared to the statistical method used for setting thresholds for differential expression (t-test noise distribution versus permutation), the number of common differentially expressed transcripts detected when each single statistical method was used to analyze MAS v.4.0 and MAS v.5.0 data, or when two statistical methods were used to analyze the same data (i.e., derived from MAS v.4.0 or MAS v.5.0) was compared. As shown in FIG. 2, the initial normalization/selection method (MAS v.4.0 or MAS v.5.0) had a greater influence on the number of common differentially expressed transcripts detected than did the statistical method used. That is, there were fewer common genes (172,177) when the two normalization/selection methods were compared, versus the number of common genes (305,332) that were evident when data obtained with a particular normalization/selection method were analyzed by two different statistical methods.

### EXAMPLE 3

### Analysis of Chromosomal Localization and Overlapping QTLs

The determination of chromosomal localization of differentially expressed known genes, and comparison to location of QTLs for AFT, were carried out using software developed by the Center for Computational Pharmacology, in the Department of Pharmacology at the University of Colorado Health Sciences Center (available from the website of the Integrated Neuroscience Initiative on Alcoholism). This software integrates Affymetrix data, QTL data, and data from the Jackson Laboratories Mouse Genome Information database. To determine the chromosomal localization of ESTs on the arrays, as well as sequence homology to known genes, and gene function, the NetAffx™ batch query tool was used (available from the Affymetrix NetAffx™ Analysis Center). If the information on the ESTs was not available from NetAffx™, manual mapping was carried out where possible using LocusLink (available from the National Center for Biotechnology Information website) and the Mouse Genome Informatics database (available from the Jackson Laboratory website). Once chromosomal localization (shown as chromosome:cM in Table 2) of the ESTs was determined, these locations were manually compared to the QTLs for AFT (Kirstein et al., J Pharmacol Exp Ther, 302:1238-1245, 2002). Genes were considered to be within the QTL if their chromosomal location was within the range of the QTL, or was within 1 cM of the marker used to define the QTL. The GenBank Accession Nos. of the mouse and human mRNA sequences corresponding to the differentially-expressed genes are shown in Table 3. The sequences in Table 3 do not necessarily correspond to that of the murine and human alleles of interest, but may be used to obtain the sequence of the alleles of interest using molecular biology techniques, which are known in the art.

After identification of transcripts through this analysis, the overall significance of the differential expression of these transcripts in HAFT-1 vs. LAFT-1 mice, and HAFT-2 versus LAFT-2 mice was determined with a 2-way ANOVA (effect of line [HAFT versus LAFT] and effect of group [HAFT-1, LAFT-1, HAFT-2, LAFT-2]). For this analysis, the raw data obtained with MAS v.4.0 and MAS v.5.0 was used. Regardless of the initial data analysis software used (MAS v.4.0 or MAS v.5.0), and regardless of the statistical method used, six genes were identified as being differentially expressed between HAFT and LAFT mice and present in QTLs for AFT or BEC2. As shown in Table 4, a two-way ANOVA performed for each of these six transcripts revealed significant main effects of line (HAFT versus LAFT), with no significant line by group interaction (HAFT-1, LAFT-1, HAFT2, LAFT-2). Thus, the six genes listed in Table 4 are contemplated to be associated with acute functional tolerance to ethanol. All but one of the differentially-expressed genes localized within the QTLs were expressed at higher levels in HAFT than in LAFT mice. Interestingly, a cluster of differentially-expressed genes located within one particular QTL (chromosome 11) were identified. There was also one differentially expressed EST (D11E17e) that was localized within the QTL on chromosome 11 (11:37), and was identified regardless of the initial data analysis or statistical method used. This EST was homologous to the S. cerevisiae gene MUM2, which plays a role in DNA replication (Davis et al., Genetics, 157:27-34, 2001). Certain other genes which map to QTL regions were also identified as being differentially expressed at the 80th percentile level by both statistical analyses, but these statistical differences were only evident with either one or the other normalization/selection procedure (MAS v.4.0 or MAS v.5.0). Thus, these genes are also contemplated to be associated with acute functional ethanol tolerance.

TABLE 2
Differentially Expressed Genes in Brains of
HAFT and LAFT mice

| Gene¹ | H1/L1 (%) | H2/L2 (%) | Localization | QTL | QTL p |
|---|---|---|---|---|---|
| Grid2 | 139 | 136 | 6:29.65 | 6:30.5-35.8 | 0.006 |
| Efnb3 | 119 | 116 | 11:40 | 11:29-43 | 0.001 |
| Grin1 | 190 | 154 | 2:12 | 2:11 | 0.001 |
| Prdx5 | 87 | 92 | 1:83.6 | 1:81.1-89.2 | 0.004 |
| Zfp179 | 159 | 171 | 11:34.5 | 11:29-43 | 0.001 |
| Tceb11 | 137 | 127 | 11:31 | 11:29-43 | 0.001 |
| Gria1² | 122 | 138 | 11:31 | 11:29-43 | 0.001 |
| Sec8³ | 120 | 162 | 6:12 | 6:10-21 | 0.001 |
| Rad50³ | 78 | 89 | 11:28.9 | 11:29-43 | 0.001 |
| Catna2³ | 85 | 82 | 6:34.2 | 6:30.5-35.8 | 0.006 |
| B2m³ | 80 | 75 | 2:69 | 2:69-71 | 0.005 |

¹The combination of permutation and MAS v.4.0 also identified solute carrier family 30 (zinc transporter) member 4, as being differentially expressed (localization, 2: 69; QTL, 2: 69-71). However, this transcript was not identified by any other combination of analyses.
²The Gria1 gene was identified as being differentially expressed only when data were initially analyzed with MAS v.4.0.
³The Sec8, Rad50, Catna2, and Btm genes were identified as being differentially expressed only when data were initially analyzed with MAS v.5.0.

TABLE 3
mRNA Sequences for Differentially Expressed Genes

| | Mouse | | Human | |
|---|---|---|---|---|
| Gene¹ | RefSeq | UniSTS | RefSeq | UniSTS |
| Grid2 | NM_008167 | D13266 | NM_001510 | AP069014 |
| Efnb3 | NM_007911 | AF025288 | NM_001406 | U66406 |
| Grin1 | NM_008169 | D10028 | NM_000832 | L13266 |
| Prdx5 | NM_012021 | AF093857 | NM_004905 | D14662 |
| Zfp179 | NM_009548 | AB013097 | NM_007148 | AF054587 |
| Tceb11 | NM_001543 | Z47088 | NM_003197 | Z47087 |
| Gria1 | NM_008165 | X57497 | NM_000827 | G15853 |
| Sec8 | NM_009148 | AF022962 | ND² | ND |
| Rad50 | NM_009012 | U66887 | NM_005732 | Z75311 |
| Catna2 | NM_009819 | D25281 | ND | ND |
| B2m | NM_009735 | X01838 | NM_004048 | AB021288 |

¹For each differentially-expressed gene, the GenBank Accession Nos. are provided for the corresponding murine and human mRNAs from the UniSTS database of the National Center for Biotechnology Information. These sequence do not necessarily correspond to that of the allele of interest, but may be used to obtain the allele of interest using molecular biology techniques which are known in the art.
²ND = not determined.

TABLE 4
Results of 2-Way ANOVA

| | | effect | | effect | |
|---|---|---|---|---|---|
| Gene | Description | of line MAS v.4.0 | | of line MAS v.5.0 | |
| Grid2 | GluRδ2 | F = 10.6 | p < 0.006 | F = 16.7 | p < 0.001 |
| | receptor | | | | |
| Efnb3 | Ephrin B3 | F = 8.4 | p < 0.010 | F = 13.7 | p < 0.002 |
| | ligand | | | | |
| Grin1 | NMDAR1 | F = 9.8 | p < 0.007 | F = 18.8 | p < 0.001 |
| Prdx5 | Peroxiredoxin | F = 6.0 | p < 0.030 | F = 10.0 | p < 0.007 |
| | 5 | | | | |
| Zfp179 | Zinc finger | F = 19.1 | p < 0.001 | F = 24.6 | p < 0.001 |
| | protein 179 | | | | |
| Tceb11 | Transcription | F = 20.2 | p < 0.001 | F = 25.3 | p < 0.001 |
| | elongation | | | | |
| | factor | | | | |

### EXAMPLE 4

### Quantitative Real-Time PCR

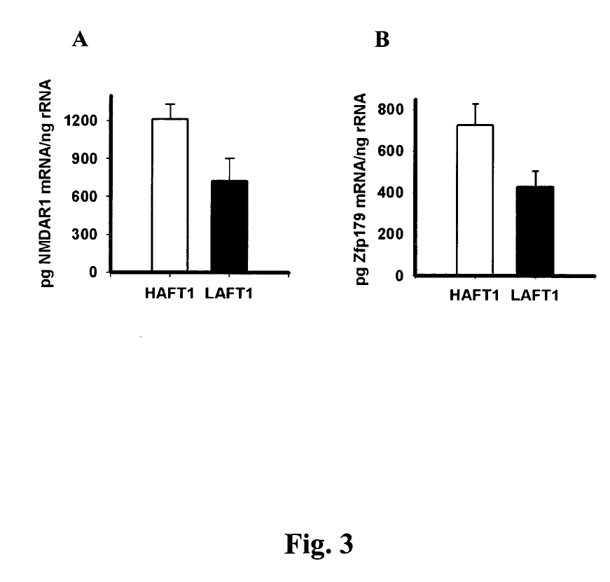

Real-time PCR was used to verify the differential expression of some of the genes indentified by microarray and QTL analyses. Briefly, quantitative real time-PCR was carried out using the ABI Prism 7700 Sequence Detection System. Sequence-specific TaqMan probes and primer sets, designed using ABI Prism 7700 sequence detection software (Primer Express, PE ABI), were used to carry out quantitative real time PCR. Probe and primer sequences are: for NR1, forward primer, 5′-GGT GGC CGT GAT GCT GTA C-3′ (SEQ ID NO:5); reverse primer, 5′-TCG CTG TTC ACC TTA AAT CGG-3′ (SEQ ID NO:6); and probe, 5′-TGC TGG ACC GCT TCA GTC CCT TTG-3′ (SEQ ID NO:7); for zinc finger protein 179, forward primer, 5′-CTG CAC TGC AGA AGA CCT GTG-3′ (SEQ ID NO:8); reverse primer, 5′-TCC GGA GGC ATT GAT TCG TA-3′ (SEQ ID NO:9); and probe, 5′-TGT GAG GGC AGA ACG TCT GCT GTT G-3′ (SEQ ID NO:10). The TaqMan probes were purchased from Perkin-Elmer, 5′-labeled with 6-carboxyfluorescein (FAM) and 3′-labeled with 6-carboxytetramethylrhodamine (TAMRA). Amplification reactions were performed in MicroAmp optical tubes in a 50 μl mix containing 8% glycerol, 1× TaqMan buffer A (500 mM KCl, 100 mM Tris-HCl, 0.1 M EDTA, 600 nM passive reference dye ROX, pH 8.3 at room temperature), 300 μM each of dATP, dGTP, dCTP and 600 μM dUTP, 5.5 mM MgCl₂, 900 nM forward primer, 300 nM reverse primer, 200 nM probe, 1.25 U AmpliTaq Gold DNA Polymerase (Perkin-Elmer), 12.5 U Moloney murine leukemia virus reverse transcriptase (Life Technologies), 20 U RNAsin (Promega) and template RNA. Thermal cycling conditions were as follows: reverse transcription was performed at 48° C. for 30 min, followed by activation of AmpliTaq Gold at 95° C. for 10 min. Subsequently 40 cycles of amplification were performed at 95° C. for 15 sec and 60° C. for 1 min. The fluorescence data were expressed as normalized reporter signal (Rn) or ΔRn. Rn was calculated by dividing the amount of reporter signal by the amount of passive reference signal. ΔRn represents the amount of normalized reporter signal minus the amount of reporter signal before PCR. The detection threshold was set above the mean baseline fluorescence determined from the first 15 cycles. Amplification reactions in which the fluorescence intensity increased above the threshold were defined as positive. A standard curve for each template was generated using a serial dilution of the template (total RNA). Quantities of template in test samples were normalized to the corresponding 18S rRNA. As shown in FIG. 3, differential expression of NMDAR1 and Zfp179 transcripts was verified by real-time PCR. Importantly, the observed magnitude of the differences in expression was similar using both microarray analysis and real-time PCR.

### EXAMPLE 5

### Determination of Transcription Factor Binding Consensus Sequences

Since the differential expression of the genes between HAFT and LAFT mice may be the result of a polymorphism in the 5′ regulatory region of certain genes, transcription factor binding sites were identified in the differentially expressed genes. Information available from both public domain and private databases was used to identify transcription factor consensus binding sequences with the assistance of Center for Computational Pharmacology resources (UCHSC). The sequences examined included: i) GenBank sequences; ii) LocusLink and RefSeq annotated entries; iii) UniGene clusters grouping all DNA and protein sequences belonging to one gene; and iv) Celera Genomics data (when no available contig information was found in the public databases). This information was used to extract the 5′-UTR sequences for further analysis. The transcription factor binding prediction was done using the TRANSFAC version 5.4 matrices from the Biobase website. At the time of this analysis, the database contained 487 binding matrices across all species. Composite regulatory element analysis was done using TransCompel version 3.0 (online version). The 5′-UTR region was obtained by extracting the sequence data from the mouse section of the Celera Genomics database using the RefSeq identifiers. The start codons were found using the GenBank RefSeq sequence information and generating 12-mer splice tags that were matched against the genomic sequence. The 1 kb and 2 kb upstream sequences were retrieved and matrix-based similarity searches against the TRANSFAC matrices were performed using the PATCH program. The core and overall matrix similarity score cutoffs were set at 0.9. The upstream sequences were searched against the TransCompel database using a maximum mismatch of 1 base pair, 30% maximum distance variation compared to the original composite element (CE) binding sites and a minimum composite score of 0.3. The composite score was calculated as follows: Composite Score=2×10^{-m1}+2×10^{-m2}+(10−g)/5 where m1, m2=number of mismatched nucleotides in the promoter core 1, 2; g=(distance between sites in the known CE)−(distance in the potential CE). Results shown are from the 2 kb upstream sequence search.

As shown in Table 5, all of the genes which were identified using both initial analysis methods and both statistical techniques, and for which information was available, were found to have the consensus sequence for CCAAT/enhancer binding protein (C/EBPα). The C/EBPα transcription factor has both constitutive and cyclic AMP-inducible activities (Wilson et al., Mol. Cell. Endocrinol., 181:27-34, 2001; and Wilson et al., Mol. Cell. Endocrinol., 188:15-20, 2002). Moreover, each of the differentially expressed genes had one or more additional transcription factor binding sites.

TABLE 5
Putative Promoters for Differentially Expressed Genes

| Gene | C/EBP-α | CREB | Myo-D | AP-1 | NF-κB |
|---|---|---|---|---|---|
| Tceb11 | + | + | | + | |
| Zfp179 | + | | + | | |
| Prdx5 | + | + | | | |
| Efnb3 | + | + | + | | + |
| Grin1 | + | + | + | | |

### EXAMPLE 6

### Comparison of Mouse and Human QTLs

Human-mouse chromosome homology maps available from the National Center for Biotechnology Information website were searched to obtain the syntenic regions of the human genome corresponding to the mouse AFT/BEC2 QTLs. These syntenic regions of the human genome were then manually aligned with the QTLs for low alcohol response in humans (Schuckit et al., Alcohol Clin Exp Res, 25:323-329, 2001). In this way, overlap of the human QTL regions with the mouse QTLs for AFT/BEC2 was estimated.

### EXAMPLE 7

### Human Study Subjects and Interviews

Study subjects were recruited for participation in the World Health Organization/International Society for Biomedical Research on Alcoholism (WHO/ISBRA) Collaborative Study on State and Trait Markers for Alcohol Use and Dependence. Subjects were excluded from the study if they manifested medical or psychiatric disorders that made them unable to respond to survey questions or if they used intravenous drugs. Participants from the study centers in Montreal, Helsinki, Sao Paulo and Sydney were included in the study and subjects of Caucasian descent were used for association analysis. After the initial screening, and before a translated version of the WHO/ISBRA Interview Schedule was administered, patients provided informed consent. On the same day as the interview, biological samples including urine and blood were collected (Glanz et al., Alcohol Clin Exp Res, 26:1047-1061, 2002).

The WHO/ISBRA Interview Schedule was adapted from the Alcohol Use and Associated Disabilities Interview Schedule developed by the National Institute on Alcohol Abuse and Alcoholism. The Interview Schedule comprised the following major sections: 1) recruitment and setting information; 2) sociodemographic background information; 3) lifetime and 30-day occurrence of medical illness including prescription medicine use; 4) frequency and quantity of beverage-specific alcohol consumption during the prior 30 days; 5) symptoms experienced during ethanol consumption, including treatment; 6) smoking history; 7) history of prescription and illicit drug use; 8) history of depression, antisocial behavior, including treatment for mental illness or emotional problems; and 9) family history of alcohol and drug problems, major depression, and antisocial behavior in first- and second-degree relatives. For use at the various clinical centers, the WHO/ISBRA Interview Schedule was translated from English into five languages: French, Finnish, German, Japanese, and Portuguese.

The WHO/ISBRA interview provides Diagnostic and Statistical Manual of Mental Disorders-Fourth Edition (DSM-IV) and International Statistical Classification of Diseases and Related Health Problems (ICD-10) diagnoses for major depression, alcohol abuse, alcohol dependence, and dependence on a number of other substances (e.g., sedatives and tranquilizers; heroin, methadone, and other opiates; stimulants and cocaine; cannabis; inhalants; hallucinogens; and anabolic steroids), antisocial personality disorder, and conduct disorder. Medical conditions queried included stomach or duodenal ulcers, hepatomegaly, hepatitis, cirrhosis, kidney disease, pancreatitis, gastritis, thyroid disease, diabetes, hyperlipidemia, tuberculosis, epilepsy, vitamin deficiencies and anemia, emphysema and other lung diseases, arthritis and osteoporosis, hypertension, heart disease, cancer, and immune system problems. The interview data also allowed for medicinal categorization of subjects who were taking medication both in the past month and in the past seven days.

The test-retest Kappa values of the major data elements appearing in the WHO/ISBRA Interview Schedule range from the low of 0.55 for items such as DSM-IV diagnosis of marijuana dependence to values of 1.0 for family history of alcohol dependence in the biological mother.

Another group of study subjects was recruited in Bethesda, Md., USA. Male and female subjects were phenotyped using: DSM-III-R questionnaire for lifetime anxiety disorders (e.g., generalized anxiety disorder, phobia, panic, obsessive-compulsive disorder); DSM-III-R questionnaire for alcohol abuse; DSM-III-R questionnaire for alcohol dependence; Tridemensional Personality Questionnaire; Eysenck Personality Questionnaire/Revised for neuroticism; Spielberg State Anxiety Questionnaire; DSM-III-R questionnaire for major depressive disorder; and Beck Depression Index. Candidates were initially screened for medical history and for alcohol, drug and medication use. Exclusion criteria were a history of brain trauma with loss of consciousness, neurological disease, or use of anxiolytics or antidepressants (Enoch et al., Physch Gen, 13:33-41, 2003).

### EXAMPLE 8

### Blood and DNA Sample Acquisition

For subjects in the WHO/ISBRA study, peripheral blood was collected at the time of the interview via standard venipuncture technique into vacutainers (20 ml blood in 15% w/v EDTA) for preparation of lymphocytes and platelets. Within two hours of collection, the platelets were prepared by centrifuging the blood samples at 700×g for 10 min at room temperature. The platelet-rich plasma layer was transferred to a fresh centrifuge tube and again centrifuged for 10 min at 700×g at room temperature. The upper platelet-rich layer was transferred to a second fresh centrifuge tube and centrifuged at 2800×g for 15 min at room temperature. The platelet pellet was recovered and stored at −70° C. until being shipped on dry ice to the Coordinating Center in Helsinki, Finland, and from there to Denver, Colo., for analysis. The lymphocyte fraction was prepared by centrifugation and frozen at −70° C. until DNA was extracted at the Assay Center in Denver Colo. Genomic DNA was extracted from the lymphocytes using the Super QUIK-GENE Rapid DNA Isolation Kit, according to the manufacturers' instructions (Analytical Genetic Testing Center).

For subjects recruited in Bethesda, Md., genomic DNA was extracted from Epstein-Barr virus immortalized lymphoblastoma cell lines (Enoch et al., 2003 supra; and Mazzanti et al., Hum Genet, 98:134-137, 1996).

### EXAMPLE 9

### Microsatellite Polymorphism Analysis

Subjects' DNA was genotyped for microsatellite polymorphisms located in an intron of the AC9 gene (i.e., second intron), in the 3′-untranslated region of the AC7 gene, and at the D16S475 marker (Foroud et al., Alcohol Clin Exp Res, 22:2035-2042, 1998) by PCR-based methods. The polymorphism in the AC9 gene is a newly-identified trinucleotide tandem repeat. Methods and compositions for detecting polymorphisms in the AC9 gene were developed by tests conducted on a sample composed of 20 randomly-selected subjects. A custom primer-pair was designed for the sense and antisense strands of the AC9 gene using the PRIMER-3 software of Rozen and Skaletsky (Whitehead Institute). The AC9 primer pair used in this analysis yields a 166 bp product from an AC9.R9 template (SEQ ID NO:1), and 168 bp product from an AC9.R10 template (sense 5′-AAC CAA GGA ACC GGG ATA TT-3′ disclosed as SEQ ID NO:11; and antisense 5′-TTT TAC GAA TCA AAG AGA TTT TTC AA-3′ disclosed as SEQ ID NO:12). The PCR products obtained from the screening samples were sequenced using an ABI Model 373 Sequencer to confirm primer specificity. Likewise, the D16S475 primer pair used in this analysis yields a 178 bp product (SEQ ID NO:3) from a D16S475.R11 allele template (sense 5′-GGT TGA CAG AGT GAG ACT C-3′ disclosed as SEQ ID NO:13; and antisense 5′-GGA ACA GAA AAT ACT GCA CG-3′ disclosed as SEQ ID NO:14). The present invention is not limited to the use of the above primers, as other oligonucleotides flanking the repeat polymorphism and yielding a PCR product less than 500 bp in length are also suitable.

The polymorphisms in the AC7 gene and methods and compositions for detecting the polymorphisms were previously described (Hellevuo et al., Neuropsych Gen, 74:95-98, 1997).

The AC7 primer pair used in this analysis yields a 203 bp product from an AC7.R7 template (SEQ ID NO:15) and a 195 bp product from an AC7.R5 template (SEQ ID NO:20) using the sense primer 5′-TTC TCC ATG GGT CAA GGA CT-3′ disclosed as SEQ ID NO:18; and the antisense primer 5′-CAT GCA CCA CCT CAA ATT CAT-3′ disclosed as SEQ ID NO:19). The present invention is not limited to the use of the published primers, as other oligonucleotides flanking the repeat polymorphism and yielding a PCR product of less than 500 bp in length are also suitable.

The primers were synthesized using an ABI 394 DNA Synthesizer and the 5′ ends were labeled with ABI's blue fluorescent phosphoramide (6-FAM). All primers were column-purified using ABI's Oligonucleotide Purification Cartridge. PCR was performed on a Perkin-Elmer GeneAmp PCR System 9700 thermocycler. Each reaction contained 50 ng of genomic DNA, 100 ng of each primer, a PCR reaction bead (Amersham) and sterile water for a total reaction volume of 25 μl. Cycling conditions were as follows: 94° C. for 12 min, 30 cycles of 94° C. for 20 sec, 55° C. for 1 min, 72° C. for 30 sec, followed by an extension at 72° C. for 1 hr and a 4° C. soak.

Positive and negative (no template) controls were included in every set of amplification reactions.

After PCR, aliquots of the samples were mixed with ABI's fluorescent Genescan-500 ROX internal lane standard and electrophoresed on an ABI Prism 310 Genetic Analyzer.

Fluorescence data were digitalized and transmitted to a Macintosh G3 computer (Apple) equipped with Genescan 672 version 3.0 and Genotyper 3.0 software. The PCR product lengths were determined based on internal standards using the Linear Southern Curve options of the analysis software.

### EXAMPLE 10

### Statistical Analysis

Where appropriate, statistical analyses were done using the chi-square test, odds ratio, multifactorial ANOVA and logistic regression with SPSS version 9.0 software. Agreement with Hardy-Weinberg equilibrium was tested for the marker alleles examined in the association studies. For this purpose, standard chi-square analysis was used to compare actual allele frequencies to the frequencies predicted by the Hardy Weinberg formula (p²+2pq+q²). The initial screening for associations between three psychiatric diagnoses and alleles or genotypes of the AC9 gene located at 16p13.3, and the D16S475 marker located at 16p13.3, was performed using the Cochran-Armitage Trend Test in the SAS® v.8.1 statistical package. The three psychiatric diagnoses considered included: major depression (MD), alcohol dependence (AD), and antisocial personality disorder (ASPD).

Specific alleles that showed a trend toward association with a given psychiatric disorder were subsequently evaluated using 2×2 chi-square analysis which provides an odds ratio and 95% confidence intervals, as shown in Table 6. The significant association between 16p13.3 locus alleles and alcohol dependence was investigated further using multiple logistic regression and by 2×2 chi-square analysis on groups stratified according to the variable of interest. The results from stratification analysis were simpler to interpret, but dividing groups by multiple variables results in fewer subjects in each group and thus a reduced ability to detect significant effects. In contrast, logistic regression allows simultaneous testing of multiple factors, and interactions between factors on a binary outcome (e.g., major depression or alcohol dependence).

As shown in Table 6, chi-square analysis showed that both the AC9.R9 and the D16S475.R11 alleles were significantly and negatively associated with alcohol dependence, and marginally and negatively associated with antisocial personality disorder. In addition, after adjusting for differences in frequencies of the AC9.R9 and D116S475.R11 alleles, chi-square analysis revealed that a significant level of linkage disequilibrium exists between these two alleles. A detailed analysis for each of the two alleles associated with alcohol dependence was performed for each separately, then the results were combined to evaluate the risk for alcohol dependence associated with possession of both the AC9.R9 and D16S475.R11 alleles.

TABLE 6
Chi-Square Analysis Results

| allele | association* | chi-square | significance | OR | 95% CI |
|---|---|---|---|---|---|
| AC9.R9 | MD (607) | 0.1 | 0.29 | 1.4 | 0.7-2.7 |
| | AD (786) | 8.9 | 0.003 | 0.6 | 0.4-0.8 |
| | ASPD (786) | 3.4 | 0.07 | 0.6 | 0.35-1.0 |
| D16S475.R11 | MD (504) | 1.0 | 0.28 | 0.7 | 0.4-1.3 |
| | AD (786) | 12 | 0.001 | 0.6 | 0.4-0.8 |
| | ASPD (786) | 3.0 | 0.06 | 0.7 | 0.4-1.0 |

*Diagnostic abbreviations include: major depression (MD), alcohol dependence (AD), and antisocial personality disorder (ASPD).

The AC9.R9 frequencies, counts, chi-square values and odds ratios were calculated for several demographic variables (Table 7). AD and ASPD were both negatively associated with the AC9.R9 allele, although in the case of ASPD the statistical significance was below the 0.05 threshold. There was no significant difference in AC9.R9 allele frequency associated with gender or major depression. In contrast, study site and ethnicity were significantly associated with the AC9.R9 allele. Participants from the Sydney location were completely devoid of the AC9.R9 allele, while Helsinki and Montreal participants differed significantly in the frequency of the AC9.R9 allele. Furthermore, blacks and Asians were less likely to possess the AC9.R9 allele as compared to whites.

Clearly, the frequency of the AC9.R9 allele differed with regard to group characteristics. In fact, the analyses of AC9.R9 suggested that study site, ASPD and/or ethnicity were the primary factors influencing the association between alcohol dependence and the AC9.R9 allele. One method for managing potentially confounding effects is to include such factors in a multifactorial logistic regression model. Thus, an initial logistic regression model was done to screen pertinent subject characteristics. Gender, AC9.R9 allele, ethnicity, study site, and ASPD were entered as independent factors in a logistic regression model with alcohol dependence as the dependent outcome. Variables were permitted to remain in the model only if they were significant at the 0.05 level. As shown in Table 8, the model 1 analysis revealed that ethnicity, ASPD and AC9.R9 were each significantly associated with alcohol dependence. To minimize the effect of population stratification, logistic regression was performed including only the white subjects in model 2. The odds ratio values for AC9.R9 remained unchanged compared to model 1 indicating that ethnicity was not confounding the association between AC9.R9 and alcohol dependence. In addition, despite the significant association between ASPD and AD, there was no significant interaction detected for AC9.9 and ASPD, indicating that each factor's association with AD is distinct.

TABLE 7
Demographic Characteristics and AC9.R9 Allele Frequencies

| characteristic (N) | ƒ[TAA]₉ | chi-square | odds ratio (95% CI) |
|---|---|---|---|
| ethnicity | | | |
| white (715) | 112 (16%) | reference | reference |
| black (11) | 1 (9%) | 0.35 (p = 0.56) | 0.54 (0.07-4.2) |
| asian (39) | 1 (2.6%) | 3.70 (p = 0.06) | 0.14 (0.02-1.0) |
| gender | | | |
| male (559) | 79 (14%) | reference | reference |
| female (227) | 39 (17%) | 0.12 (p = 0.28) | 1.3 (0.8-1.9) |
| lifetime AD | | | |
| positive (432) | 50 (12%) | 8.9 (p = 0.003) | 0.6 (0.4-0.8) |
| negative (354) | 68 (19%) | reference | reference |
| ASPD | | | |
| positive | 17 (10%) | 3.9 (p = 0.07) | 0.6 (0.35-1.0) |
| negative | 101 (16%) | reference | reference |
| MD | | | |
| positive | 26 (16%) | 0.12 (p = 0.74) | 1.1 (0.67-1.7) |
| negative | 92 (14.8%) | reference | reference |
| study site | | | |
| Montreal (453) | 85 (19%) | reference | reference |
| Helsinki (89) | 33 (37%) | 14.0 (p < 0.0002) | 4.2 (2.4-4.6) |
| Sydney (244) | 0 (0%) | NA* | NA* |

*NA = not applicable as no subjects from this site possessed the AC9.R9 allele.

TABLE 8
Logistic Regression Analysis for AC9.R9 and Alcohol Dependence
(Dependent Variable = Alcohol Dependence)

| variable | B | SE | χ² | df | sig. | OR | lower | upper |
|---|---|---|---|---|---|---|---|---|
| model 1* | | | | | | | | |
| AC9.R9 | −0.639 | 0.223 | 8.206 | 1 | 0.004 | 0.528 | 0.341 | 0.817 |
| ASPD | 2.518 | 0.306 | 67.511 | 1 | 0.00000 | 12.404 | 6.803 | 22.617 |
| black | −0.785 | 0.703 | 1.246 | 1 | 0.264 | 0.456 | 0.115 | 1.810 |
| asian | −3.116 | 0.750 | 17.237 | 1 | 0.00003 | 0.044 | 0.010 | 0.193 |
| white | reference | — | — | — | — | — | — | — |
| model 2** | | | | | | | | |
| AC9.R9 | −0.677 | 0.224 | 9.161 | 1 | 0.002 | 0.508 | 0.328 | 0.788 |
| ASPD | 2.416 | 0.303 | 63.581 | 1 | 0.00000 | 11.200 | 6.185 | 20.283 |

*model 1: white (n = 715), black (n = 11), and Asian (n = 39); R² = 0.20.
**model 2: white (n = 715); R² = 0.15.

The D16S475.R11 marker and the AC9.R9 allele are located within 2 cM of one another on the short arm of chromosome 16. Chi-square analysis of the two markers demonstrated that D16S475.R11 and AC9.R9 were in linkage disequilibrium (mean frequency difference of 17.45%, 95% CI=8.2−26.7, p<0.0001). The D16S475.R11 allele frequencies, counts, chi-square values and odds ratios were calculated for several demographic variables. As shown in Table 9, there was no significant association between D16S475.R11 allele frequency and gender, ethnicity or depression. In contrast, significant associations between AD, ASPD and the D16S475.R11 allele, and a marginally significant association between study site and the D16S475.R11 allele were present. In particular, AD and ASPD were both negatively associated with the D16S475.R11 allele, and subjects at the Montreal site were significantly more likely to exhibit the D16S475.R11 allele compared to subjects in Helsinki. However, there was not a significant difference in D16S475.R11 allele frequencies when comparing Sydney to Helsinki.

TABLE 9
Demographic Characteristics and
D16S475.R11 Allele Frequencies

| characteristic (N) | ƒ[GA]₁₁ | chi-square | odds ratio (95% CI) |
|---|---|---|---|
| ethnicity | | | |
| white (581) | 200 (34%) | reference | reference |
| black (10) | 4 (40%) | 0.14 (p = 0.74) | 1.3 (0.4-4.5) |
| Asian (33) | 14 (42%) | 0.87 (p = 0.35) | 1.4 (0.7-2.9) |
| gender | | | |
| male (484) | 174 (36%) | reference | reference |
| female (159) | 50 (31%) | 1.1 (p = 0.30) | 0.8 (0.6-1.2) |
| lifetime AD | | | |
| positive (347) | 100 (29%) | 12.0 (p = 0.001) | 0.6 (0.4-0.8) |
| negative (296) | 124 (42%) | reference | reference |
| ASPD | | | |
| positive (117) | 32 (27%) | 3.5 (p = 0.06) | 0.7 (0.4-1.0) |
| negative (526) | 192 (37%) | reference | reference |
| MD | | | |
| positive (123) | 38 (31%) | 1.0 (p = 0.31) | 0.8 (0.5-1.2) |
| negative (520) | 186 (36%) | reference | reference |
| study site | | | |
| Helsinki (110) | 48 (44%) | reference | reference |
| Montreal (306) | 97 (32%) | 5.0 (p = 0.025) | 0.6 (0.38-0.94) |
| Sydney (227) | 79 (35%) | 2.4 (p = 0.117) | 0.7 (0.43-1.1) |

An initial logistic regression model for the purpose of screening pertinent subject characteristics was performed. Gender, D16S475.R11 allele, ethnicity, study site, and ASPD were entered as independent factors in a logistic regression model and AD was entered as the dependent factor (outcome). Independent variables remained in the model if the factor's effect was associated with significance equal to or better than 0.05. The results from the screening step led to model 1 of Table 10. The model 1 analysis revealed that ethnicity, ASPD and D16S475.R11 were each significantly associated with AD. To minimize the possibility of population stratification, logistic regression was performed including only the white subjects in model 2 of Table 10. The odds ratio values and significance level for the D16S475.R11 allele and AD remained unchanged, indicating that ethnicity was not playing a large role in the association between D16S475.R11 and AD. In addition, there was no significant interaction between D16S475.R11 and ASPD (independent factors), further confirming that each factor's association with AD is distinct.

TABLE 10
Logistic Regression Analysis for D16S475.R11 and Alcohol Dependence
(Dependent Variable = Alcohol Dependence)

| variable | B | SE | χ² | df | sig. | OR | lower | upper |
|---|---|---|---|---|---|---|---|---|
| model 1* | | | | | | | | |
| D16S475.R11 | −0.508 | 0.185 | 7.515 | 1 | 0.006 | 0.602 | 0.418 | 0.865 |
| ASPD | 2.369 | 0.345 | 47.179 | 1 | 0.00000 | 10.690 | 5.437 | 21.019 |
| black | −1.053 | 0.752 | 1.961 | 1 | 0.161 | 0.349 | 0.080 | 1.523 |
| Asian | −3.392 | 1.014 | 11.203 | 1 | 0.001 | 0.034 | 0.005 | 0.245 |
| white | reference | — | — | — | — | — | — | — |
| model 2** | | | | | | | | |
| D16S475.R11 | −0.505 | 0.187 | 7.282 | 1 | 0.007 | 0.603 | 0.418 | 0.817 |
| ASPD | 2.346 | 0.345 | 46.230 | 1 | 0.00000 | 10.448 | 5.312 | 20.548 |

*model 1: white (n = 581), black (n = 10), and Asian (n = 35); R² = 0.149.
**model 2: white (n = 581); R² = 0.134.

The two marker alleles located at the 16p13.3 locus, D16S475.R11 and AC9.R9, share several characteristics. For instance, these two alleles have similar odds ratios and significance values for association with alcohol dependence. In addition, each marker showed marginal association with ASPD and both alleles were significantly more common in the study subjects from Helsinki as compared to Montreal and Sydney. In order to examine the combined effect of both alleles on the risk for AD, each of the two alleles was entered in a logistic regression model as an independent factor with AD as the outcome. Then, to examine the possibility of an interaction between D16S475.R11 and AC9.R9, an interaction term was entered into the model. The results from this analysis provided in Tables 11 and 12, showed the effect on AD from each allele separately and when combined. The D16S475.R11 and AC9.R9 alleles showed a significant protective influence on AD through a main effect from each allele, but not through any significant interaction between the D16S475.R11 and AC9.R9 alleles. Among Caucasian subjects from the Montreal study site, those individuals that possess both the AC9.R9 and D16S475.R11 alleles, are at a 5-6 times lower risk for development of AD as compared to subjects without both the AC9.R9 and D16S475.R11 alleles. Thus, the association between the AC9.R9 and D16S475.R11 alleles and alcohol dependence is a protective one. This is in contrast to the results obtained by Foroud and colleagues (Foroud et al., Alcohol Clin Exp Res, 22:2035-2042, 1998).

TABLE 11
Association Analysis for AC9.R9 and D16S475.R11 Allele Pairs*

| | neither | | | AC9.R9 | |
|---|---|---|---|---|---|
| Value | allele | D16S475.R11 only | D16S475.R11 + AC9.R9 | only | Total |
| Never AD | | | | | |
| Count | 37 | 29 | 16 | 24 | 106 |
| Expected | 54.7 | 23.9 | 9.3 | 18.1 | 106 |
| % in AD | 34.9% | 27.4% | 15.1% | 22.6% | 100% |
| % in pair | 26.1% | 46.8% | 66.7% | 51.1% | 38.5% |
| % of total | 13.5% | 10.5% | 5.8% | 8.7% | 38.5% |
| adjusted | −4.4 | 1.5 | 3.0 | 1.9 | |
| residual | | | | | |
| Lifetime AD | | | | | |
| Count | 105 | 33 | 8 | 23 | 169 |
| Expected | 87.3 | 38.1 | 14.7 | 28.9 | 169 |
| % in AD | 62.1% | 19.5% | 4.7% | 16.3% | 100% |
| % in pair | 73.9% | 53.2% | 33.3% | 48.9% | 61.5% |
| % of total | 38.2% | 12.0% | 2.9% | 8.4% | 61.5% |
| adjusted | 4.4 | −1.5 | −3.0 | −1.9 | |
| residual | | | | | |

*White subjects from Montreal,
Pearson chi-square = 22.24,
df = 3,
p = 0.00006.

TABLE 12
Logistic Regression Analysis of Association between
the AC9.R9 and D16S475.R11 Alleles and Alcohol Dependence

| Alleles | D16S475.R11 present | D16S475.R11 absent |
|---|---|---|
| AC9.R9 present | OR = 0.185 | OR = 0.427 |
| | 95% CI = 0.087-0.389 | 95% CI = 0.249-0.733 |
| | nonAD = 16 + AD = 8 | nonAD = 24 + AD = 23 |
| AC9.R9 absent | OR = 0.434 | OR = 1 |
| | 95% CI = 0.261-0.723 | nonAD = 137 + AD = 105 |
| | nonAD = 29 + AD = 33 | |

Prior to this analysis, there had been conflicting reports regarding a positive association between the 16p13.3 locus and alcoholism severity. During the development of the present invention, the lack of a positive correlation between two 16p13.3 markers and alcohol dependence was observed. In fact, the present study documents a negative association between alcohol dependence and the AC9.R9 and D16S475.R11 alleles. The detection of a negative association in the present work is contemplated to be the result of the case control association study design employed herein, as contrasted with the linkage study design employed by others, since the association study design is more sensitive in finding an influence of genes on phenotype.

The AC9.R9 and the D16S475.R11 alleles were compared with regard to the actual symptomology that was used to determine the DSM-IV alcohol dependence diagnosis. As shown in Table 13 below, the comparison indicates that there is substantial difference in the symptom “profile” of those individuals with the D16S475.R11 allele compared to individuals with the AC9.R9 allele. Thus, the two markers are contemplated to be associated with different subgroups of alcohol dependent subjects.

TABLE 13
Chi-Square Analysis for Associations between Diagnostic Symptoms
of AD and the AC9.R9 and D16S475.R11 Alleles

| | D16S475.R11 Allele | | | AC9.R9 Allele | | |
|---|---|---|---|---|---|---|
| Symptom | OR | 95% CI | sig. | OR | 95% CI | sig. |
| tried but couldn't cut down | 0.65 | 0.43-0.98 | 0.040 | | | NS |
| drink despite decision against | 0.61 | 0.42-0.900 | 0.012 | | | NS |
| drink more than intended | 0.60 | 0.38-0.95 | 0.027 | | | NS |
| restless when not drinking | 0.55 | 0.35-0.86 | 0.008 | | | NS |
| drink despite depression | 0.57 | 0.37-0.87 | 0.008 | | | NS |
| want to drink badly | 0.60 | 0.39-0.95 | 0.028 | | | NS |
| strong urge to drink | 0.59 | 0.40-0.87 | 0.007 | | | NS |
| drink more for same effect | | | NS | 0.55 | 0.34-0.90 | 0.015 |
| drinking interferes with family | | | NS | 0.58 | 0.35-0.97 | 0.036 |
| fighting while/after drinking | | | NS | 0.32 | 0.10-1.0 | 0.040 |
| arrest unrelated to traffic | | | NS | 0.41 | 0.21-0.81 | 0.008 |

### EXAMPLE 11

### Further Statistical Analysis

Phenotypic associations with the AC9.R9 allele were investigated in the WHO/ISBRA population. To control for stratification due to race/ethnicity, analyses were carried out using data from white (Caucasian) subjects only. To manage other potentially confounding influences on phenotypic associations with the AC9.R9 allele, various factors (a total of 32 variables, Table 15) were entered as independent factors in a logistic regression analysis with alcohol consumption (>15 g/day) as the dependent outcome. Alcohol consumption was chosen as the dependent variable because a prior analysis of AC9.R9 allele frequencies demonstrated that the frequency of the AC9.R9 allele was 50% greater in the segment of the population drinking <15 g/day compared to the segment of the population drinking >15 g/day (p=0.03). For the logistic analysis, data from Caucasian subjects from four study sites (Helsinki, Finland; Montreal, Canada; São Paulo, Brazil; and Sydney, Australia) were used. Variables were permitted to remain in the final model only if they were significant at the p<0.05 level. The analysis demonstrated that the AC9.R9 allele was significantly (p=0.024) and negatively associated with drinking more than 15 g of alcohol/day. The presence of this allele decreased the likelihood of drinking more than 15 g of alcohol/day by 1.7-fold. When AC7 and AC9 alleles were not included in the final analysis, another factor associated with a decreased likelihood of drinking more than 15 g of alcohol/day was gender (females are less likely to drink more than 15 g of alcohol/day than males). On the other hand, smoking and lifetime alcohol abuse or dependence were associated with higher levels of alcohol drinking, as were elevated levels of carbohydrate-deficient transferrin and gamma-glutamyl transferase, both of which are biological markers of high alcohol consumption.

TABLE 14
Association of the AC9.R9 Allele with Alcohol Consumption

| Group | Number | Estimate | Odds Ratio¹ | P Value |
|---|---|---|---|---|
| Helsinki males | 90 | 0.616 | 3.43 | 0.007 |
| Montreal males | 204 | 0.073 | 1.16 | 0.682 |
| Montreal females | 211 | 0.544 | 2.97 | 0.004 |

¹The odds ratio indicates the likelihood of having the AC9.R9 allele when the subject consumed less that 15 g of alcohol per day.

TABLE 15
Variables used for Further Logistic Regression Analysis

| Symbol | Variable |
|---|---|
| GENDER | Sex |
| AGE | Age (years) |
| EXERREG | Regular Exercise |
| SMOKTYPE | Smoking recode |
| DRINKERS | Drinking recode |
| GTOT_30D | Alcohol consumed (g) in last 30 days |
| CONQUART | Alcohol consumption in quartiles (gm/day) |
| AD_LIFE | Lifetime alcohol dependence |
| AB_LIFE | Lifetime alcohol abuse |
| FHX1AD | Family history of alcohol dependence in |
| | 1st degree relative, without clustering |
| FHX1ADC | Family history of alcohol dependence in |
| | 1st degree relative, with clustering |
| DEPRES4F | DSM-IV major depression during subject's lifetime |
| FHX_DEP1 | Family history of depression in 1st degree relative |
| ANTIDP30 | Used any antidepressant in last month |
| OTHER30 | Used medication other than antidepressants in last month |
| ANXIETY | Ever seek treatment for anxiety |
| CONDUCT | Conduct disorder (DSM-IV) |
| ANTISOC | Antisocial personality disorder (DSM-IV) |
| MJA_LIFE | Lifetime marijuana abuse |
| AC.VII.G | AC-VII genotypes |
| AC.VIIA1 | AC-VII allele (AACA) 5 |
| AC.VIIA2 | AC-VII allele (AACA) 6 |
| AC.VIIA3 | AC-VII allele (AACA) 7 (AC7.R7) |
| CDT | carbohydrate-deficient transferrin level |
| GGT | gamma-glutamyl transferase level |
| CSF_AC-4 | CSF-stimulated AC activity (quartiles) |
| FOR_AC | Forskolin-stimulated AC activity |
| CSF_AC | CSF-stimulated AC activity |
| FOR_AC_4 | Forskolin-stimulated AC activity (quartiles) |
| AC.IX.G | AC-IX Genotypes |
| AC.IX.A1 | AC-IX allele (TAA) 8 |
| AC.IX.A2 | AC-IX AC-IX allele (TAA) 9 |
| AC.IX.A3 | AC-IX allele (TAA) 10 |
| AC.IX.A4 | AC-IX allele (TAA) 11 |
| AC.IX.A5 | AC-IX allele (TAA) 12 |
| AC.IX.A6 | AC-IX allele (TAA) 13 |

The predictive power of the AC9.R9 allele for alcohol consumption using ROC analysis is illustrated in FIG. 6.

The AC7 gene is located on the opposite arm of chromosome 16 from the AC9 gene, at 16q12.2. The AC7.R7 frequencies, counts, chi-square values and odds ratios were calculated for several demographic variables in the WHO/ISBRA study population. As for AC9.R9, AC7.R7 allele frequency differed among ethnic groups. Therefore, for analyses, only data from Caucasian subjects was used. In this population, there was a marginally significant (χ²=3.3, p<0.07) negative association of the AC7.R7 allele with alcohol dependence.

Logistic regression analysis, using the variables shown in Table 15, was also carried out to determine independent associations of phenotype and the AC7.R7 allele in Caucasian subjects from the four study sites (Helsinki, Montreal, São Paulo, Sydney). The dependent outcome in this analysis was the presence of the AC7.R7 allele and variables were permitted to remain in the final model only if they were significant at the p<0.05 level. The results showed that, in the total population, individuals with a lifetime history of alcohol dependence (DSM-IV) are 1.3 times less likely (p<0.039) to have the AC7.R7 allele. This effect is most pronounced in male subjects (O.R. 0.73, p<0.05), and in fact the association was not statistically significant when female subjects were analyzed separately. In the total population, there were no other phenotypic variables found to show statistically significant associations with the presence of the AC7.R7 allele.

Phenotypic associations of alcohol abuse and alcohol dependence with the AC7.R5 allele were analyzed in the population comprising the Caucasian individuals recruited in Bethesda, Md. and the WHO/ISBRA Montreal population, in which alcohol abuse (DSM-IIIBR, DSM-IV) could be accurately ascertained and distinguished from alcohol dependence (DSM-IIIBR, DSM-IV). The AC7.R5 allele frequencies and counts in this population, chi-square values and odds ratios were calculated for several demographic variables. There was a marginally significant (p<0.06) positive allelic association with alcohol dependence, and alcohol abuse was significantly (χ² 10.6, p<0.001) positively associated with the presence of the AC7.R5 allele.

Logistic regression analysis was carried out to determine independent phenotypic associations with the AC7.R5 allele in this population. Analyses in which lifetime alcohol abuse or AC7.R5 were used as the dependent outcome variables were performed. The independent variables used in the analyses were: i) for alcohol abuse as outcome: age, gender, alcohol dependence, depression, anxiety, AC7.R5, AC7.R6, AC7.R7, AC9.R9, AC9.R10, AC9.R11, AC9.R12; and ii) for AC7.R5 as outcome: the same variables plus alcohol abuse and without AC7.R5. The results showed that individuals with the AC7.R5 allele were nearly 2 times more likely (OR=1.97) to have a lifetime history of alcohol abuse than those without the AC7.R5 allele (χ²=12.22, p<0.0005). When AC7.R5 was used as the outcome variable, logistic regression analysis again indicated that subjects with a history of alcohol abuse are nearly twice as likely to have the AC7.R5 allele as those without a history of alcohol abuse (O.R. 1.78, χ²=10.49, p<0.001).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in molecular biology, genetics, or related fields are intended to be within the scope of the following claims.

What is claimed is:

1. A method of identifying individuals predisposed to alcohol tolerance and dependence comprising:
a) providing a sample from a human subject; and
b) screening said sample for a biomarker selected from the group consisting of Grid2, Efnb3, Grin1, Zfp179, Tceb11, Gria1, Sec8, Prdx5, Rad50, Catna2, and B2m, wherein said biomarker is correlated with a predisposition to alcohol tolerance and dependence.

2. The method of claim 1, wherein said biomarker is selected from the group consisting of Grid2, Efnb3, Grin1, Zfp179, Tceb11, Gria1, and Sec8, and wherein said biomarker is associated with transcriptional upregulation.

3. The method of claim 1, wherein said biomarker is selected from the group consisting of Prdx5, Rad50, Catna2, and B2m, and wherein said biomarker is associated with transcriptional downregulation.

4. The method of claim 1, wherein said screening is accomplished by quantitating the mRNA or protein level of said biomarker.

5. The method of claim 4, wherein said mRNA level is quantitated by a method selected from the group consisting of real time polymerase chain reaction, and gene expression array.

6. The method of claim 1, wherein said screening is accomplished by detection of a polymorphism in said biomarker.

7. The method of claim 6, wherein said detection is accomplished using at least one technique selected from the group consisting of polymerase chain reaction, heteroduplex analysis, single stand conformational polymorphism analysis, ligase chain reaction, comparative genome hybridisation, Southern blotting and sequencing.

8. A method of identifying individuals at reduced risk of developing alcohol dependence, comprising:
a) providing a sample from a human subject; and
b) screening said sample for at least one biomarker of the 16p13.3 locus or the 16q12.2 locus, wherein said biomarker is correlated with a reduced risk of developing at least one symptom of alcohol dependence and/or with reduced alcohol consumption.

9. The method of claim 8, wherein said biomarker is selected from the group consisting of the AC9 gene, the AC7 gene, and markers in linkage disequilibrium thereof, and wherein said screening is accomplished by detection of a polymorphism in said biomarker.

10. The method of claim 9, wherein said biomarker is the AC9 gene, and said polymorphism is a [TAA]₉ repeat in an intron of said AC9 gene.

11. The method of claim 9, wherein said biomarker is the AC7 gene, and said polymorphism is an [AACA]₇ repeat in the 3′-untranslated region of said AC7 gene.

12. The method of claim 8, wherein said at least one symptom comprises a symptom selected from the group consisting of alcohol tolerance, alcohol withdrawal, heavy alcohol consumption, persistent desire to reduce alcohol consumption, considerable time spent in acquiring, using or recovering from alcohol, alcohol-related reduction in social, occupational, or recreational activities, and continued alcohol use despite alcohol-related physical or psychological problems.

13. The method of claim 9, wherein said detection is accomplished using at least one technique selected from the group consisting of polymerase chain reaction, heteroduplex analysis, single stand conformational polymorphism analysis, ligase chain reaction, comparative genome hybridisation, Southern blotting and sequencing.

14. A method of identifying individuals at increased risk for alcohol abuse, comprising:
a) providing a sample from a human subject; and
b) screening said sample for at least one biomarker of the 16q12.2 locus, wherein said biomarker is correlated with increased risk of developing at least one symptom of alcohol abuse.

15. The method of claim 14, wherein said biomarker is selected from the group consisting of the AC7 gene and a marker in linkage disequilibrium with the AC7 gene, and wherein said screening is accomplished by detection of a polymorphism in said marker.

16. The method of claim 15, wherein said biomarker is the AC7 gene, and said polymorphism is an [AACA]₅ repeat in the 3′-untranslated region of said AC7 gene.

17. The method of claim 14, wherein said at least one symptom comprises a symptom selected from the group consisting of recurrent alcohol use resulting in failure to fulfill major obligations, recurrent alcohol use in physically hazardous situations, recurrent alcohol-related legal problems, and continued alcohol use despite alcohol-related social problems.

18. The method of claim 15, wherein said detection is accomplished using at least one technique selected from the group consisting of polymerase chain reaction, heteroduplex analysis, single stand conformational polymorphism analysis, ligase chain reaction, comparative genome hybridisation, Southern blotting and sequencing.

19. A kit for determining whether a subject is at a reduced or increased risk for developing alcohol abuse and/or dependence, comprising:
a) at least one reagent capable of specifically detecting at least one polymorphism in an allele of a gene selected from the group consisting of AC9, AC7, and genes in linkage disequilibrium thereof; and
b) instructions for determining whether a subject is at a reduced or increased risk of developing abuse and/or alcohol dependence.

20. The kit of claim 19, wherein said at least one polymorphism is a microsatellite repeat polymorphism.

21. The kit of claim 19, wherein said at least one reagent comprises a nucleic acid probe that hybridizes under stringent conditions to a nucleic acid sequence selected from the group consisting of the coding strand of said gene, and the noncoding strand of said gene.

22. The kit of claim 19, wherein said at least one reagent comprises a sense primer and an antisense primer flanking said at least one polymorphism in said allele.

23. The kit of claim 22, wherein at least one of said primers comprises a fluorescent tag.

24. A method of screening compounds, comprising:
a) providing:
i) at least one allele of a gene selected from the group consisting of Grid2, Efnb3, Grin1, Zfp179, Tceb11, Gria1, Sec8, Prdx5, Rad50, Catna2, B2m, AC9, and AC7, or a cell transfected with said at least one allele of said gene, and
ii) one or more test compounds; and
b) contacting said gene or said cell with said test compound; and
c) detecting a change in expression of said gene in the presence of said test compound relative to the absence of said test compound.

25. The method of claim 24, wherein said detecting comprises detecting RNA transcribed from said gene.

26. The method of claim 24, wherein said detecting comprises detecting a polypeptide encoded by said gene.

27. The method of claim 24, wherein said gene is contained within an eukaryotic cell when contacting said gene with said test compound.

28. The method of claim 24, wherein said gene is contained within an in vitro transcription reaction when contacting said gene with said test compound.


SEQUENCE LISTING (20 sequences)

SEQ ID NO: 1 - DNA, 166 bp, Homo sapiens
aaccaaggaaccgggatattaaattacttctctaggtcatgcagctagtgtgcggtgggctaaatgtacaatccccctaataataataataataataataataacttacaacaacagcatattgaaataagaaaaaaaatttgaaaaatctctttgattcgtaaaa

SEQ ID NO: 2 - DNA, 27 bp, Homo sapiens
taataataataataataataataataa

SEQ ID NO: 3 - DNA, 175 bp, Homo sapiens
ggttgacagagtgagactccatcaaaaaaaagaaagaaagaaacacagaaagagagagagacgaaagaaagagagagagagagagagagagaaagaaagaaagaaagaaagaaagaaagaaagaaagaaagaaagaaagaaagaaagaaagaaagttccgtgcagtattttctgt

SEQ ID NO: 4 - DNA, 22 bp, Homo sapiens
gagagagagagagagagagaga

SEQ ID NO: 5 - DNA, 19 bp, Artificial Sequence
  Synthetic
ggtggccgtgatgctgtac

SEQ ID NO: 6 - DNA, 21 bp, Artificial Sequence
  Synthetic
tcgctgttcaccttaaatcgg

SEQ ID NO: 7 - DNA, 24 bp, Artificial Sequence
  Synthetic
tgctggaccgcttcagtccctttg

SEQ ID NO: 8 - DNA, 21 bp, Artificial Sequence
  Synthetic
ctgcactgcagaagacctgtg

SEQ ID NO: 9 - DNA, 20 bp, Artificial Sequence
  Synthetic
tccggaggcattgattcgta

SEQ ID NO: 10 - DNA, 25 bp, Artificial Sequence
  Synthetic
tgtgagggcagaacgtctgctgttg

SEQ ID NO: 11 - DNA, 20 bp, Artificial Sequence
  Synthetic
aaccaaggaaccgggatatt

SEQ ID NO: 12 - DNA, 26 bp, Artificial Sequence
  Synthetic
ttttacgaatcaaagagatttttcaa

SEQ ID NO: 13 - DNA, 19 bp, Artificial Sequence
  Synthetic
ggttgacagagtgagactc

SEQ ID NO: 14 - DNA, 20 bp, Artificial Sequence
  Synthetic
ggaacagaaaatactgcacg

SEQ ID NO: 15 - DNA, 203 bp, Homo sapiens
ttctccatgggtcaaggacttccttacaatttctcctgagttaacttttgtgaaaataatacctaaggttttctggcttattgaggaaatttcctaacaaacaaacaaacaaacaaacaaacagaagagaagatcattaaccactgtatactttgtgtatataataggtcagtgtaaagaaatatgatttgaggtggtgcatg

SEQ ID NO: 16 - DNA, 28 bp, Homo sapiens
aacaaacaaacaaacaaacaaacaaaca

SEQ ID NO: 17 - DNA, 20 bp, Homo sapiens
aacaaacaaacaaacaaaca

SEQ ID NO: 18 - DNA, 20 bp, Artificial Sequence
  Synthetic
ttctccatgggtcaaggact

SEQ ID NO: 19 - DNA, 21 bp, Artificial Sequence
  Synthetic
catgcaccacctcaaattcat

SEQ ID NO: 20 - DNA, 195 bp, Homo sapiens
ttctccatgggtcaaggacttccttacaatttctcctgagttaacttttgtgaaaataatacctaaggttttctggcttattgaggaaatttcctaacaaacaaacaaacaaacagaagagaagatcattaaccactgtatactttgtgtatataataggtcagtgtaaagaaatatgatttgaggtggtgcatg